(19) United States
(12) Patent Application Publication
(10) Pub. No.: US 2026/0364295 A1
(43) Pub. Date: Nov. 19, 2026

(54) PATIENT INTERFACE AND CUSHION THEREFOR

(71) Applicant: ResMed Pty Ltd, Bella Vista, New South Wales (AU)

(72) Inventors: Michiel KOOIJ, Sydney (AU); Rupert Christian SCHEINER, Sydney (AU); Luke Andrew STANISLAS, Sydney (AU); Stewart Joseph WAGNER, Hawkesbury (AU); Chiew Khuen WONG, Sydney (AU)

(21) Appl. No.: 19/166,012

(22) PCT Filed: Mar. 18, 2024
(86) PCT No.: PCT/AU2024/050235

§ 371 Date: Sep. 16, 2025

(30) Foreign Application Priority Data
Mar. 17, 2023 (AU) 2023900733

(51) Int. Cl. A61M 16/06

(52) U.S. Cl. CPC A61M 16/0622; A61M 16/0683; A61M 2205/0216; A61M 2207/10

(57) ABSTRACT

A cushion module for a patient interface, comprising: a plenum chamber; a seal-forming structure; a chassis portion and a membrane portion together at least partially forming the plenum chamber, the membrane portion being attached to the chassis portion and forming the seal-forming structure, the chassis portion and membrane portion being formed separately; wherein the membrane portion is attached to the chassis portion in an inverted position in which the membrane portion is positioned on an anterior side of the chassis portion, the membrane portion then being inverted to an in-use position in which the membrane portion is positioned on a posterior side of the chassis portion.

### 1 BACKGROUND OF THE TECHNOLOGY

### 1.1 Field of the Technology

The present technology relates to one or more of the screening, diagnosis, monitoring, treatment, prevention and amelioration of respiratory-related disorders. The present technology also relates to medical devices or apparatus, and their use.

### 1.2 Description of the Related Art

### 1.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD) and Chest wall disorders.

Obstructive Sleep Apnea (OSA), a form of Sleep Disordered Breathing (SDB), is characterised by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem. See U.S. Pat. No. 4,944,310 (Sullivan).

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterised by repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload. See U.S. Pat. No. 6,532,959 (Berthon-Jones).

Respiratory failure is an umbrella term for respiratory disorders in which the lungs are unable to inspire sufficient oxygen or exhale sufficient CO₂ to meet the patient's needs. Respiratory failure may encompass some or all of the following disorders.

A patient with respiratory insufficiency (a form of respiratory failure) may experience abnormal shortness of breath on exercise.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

A range of therapies have been used to treat or ameliorate such conditions. Furthermore, otherwise healthy individuals may take advantage of such therapies to prevent respiratory disorders from arising. However, these have a number of shortcomings.

### 1.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 1.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube. In some forms, the comfort and effectiveness of these therapies may be improved.

### 1.2.2.2 Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate” that is held approximately constant throughout the respiratory cycle. The treatment flow rate is nominally set to exceed the patient's peak inspiratory flow rate. HFT has been used to treat OSA, CSR, respiratory failure, COPD, and other respiratory disorders. One mechanism of action is that the high flow rate of air at the airway entrance improves ventilation efficiency by flushing, or washing out, expired CO₂ from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

Another form of flow therapy is long-term oxygen therapy (LTOT) or supplemental oxygen therapy. Doctors may prescribe a continuous flow of oxygen enriched gas at a specified oxygen concentration (from 21%, the oxygen fraction in ambient air, to 100%) at a specified flow rate (e.g., 1 litre per minute (LPM), 2 LPM, 3 LPM, etc.) to be delivered to the patient's airway.

### 1.2.2.3 Supplementary Oxygen

For certain patients, oxygen therapy may be combined with a respiratory pressure therapy or HFT by adding supplementary oxygen to the pressurised flow of air. When oxygen is added to respiratory pressure therapy, this is referred to as RPT with supplementary oxygen. When oxygen is added to HFT, the resulting therapy is referred to as HFT with supplementary oxygen.

### 1.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

Another form of therapy system is a mandibular repositioning device.

### 1.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

Certain other mask systems may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth.

Certain masks may be uncomfortable or impractical for the present technology if they require a patient to insert a portion of a mask structure in their mouth to create and maintain a seal via their lips.

Certain masks may be impractical for use while sleeping, e.g. for sleeping while lying on one's side in bed with a head on a pillow.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

As a consequence of these challenges, some masks suffer from being one or more of obtrusive, aesthetically undesirable, costly, poorly fitting, difficult to use, and uncomfortable especially when worn for long periods of time or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. This discomfort may lead to a reduction in patient compliance with therapy. This is even more so if the mask is to be worn during sleep.

CPAP therapy is highly effective to treat certain respiratory disorders, provided patients comply with therapy. If a mask is uncomfortable, or difficult to use a patient may not comply with therapy. Since it is often recommended that a patient regularly wash their mask, if a mask is difficult to clean (e.g., difficult to assemble or disassemble), patients may not clean their mask and this may impact on patient compliance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

Some patient interfaces of the prior art comprise a cushion module having a rigid shell with a predefined shape based on the anthropometrics of a notional person in the middle of a selected size range. As a result, the seal and comfort success with a given size of cushion module may be strongly related to the correlation between the patient's anthropometrics and those of the notional person on which the design is based. This may result in the need to “fit” the patient (possibly with assistance from a suitably qualified professional) to a particular size of cushion module and/or a particular type of interface. This may also result in the need to manufacture a range of different size cushion modules to ensure that a broad range of patients can find a size with suits them.

### 1.2.3.1.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material, for example silicone, positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

Another form of seal-forming structure may use adhesive to achieve a seal. Some patients may find it inconvenient to constantly apply and remove an adhesive to their face.

A range of patient interface seal-forming structure technologies are disclosed in the following patent applications, assigned to ResMed Limited: WO 1998/004,310; WO 2006/074,513; WO 2010/135,785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Limited has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications, assigned to ResMed Limited, describe examples of nasal pillows masks: International Patent Application WO2004/073,778 (describing amongst other things aspects of the ResMed Limited SWIFT™ nasal pillows), US Patent Application 2009/0044808 (describing amongst other things aspects of the ResMed Limited SWIFT™ LT nasal pillows); International Patent Applications WO 2005/063,328 and WO 2006/130,903 (describing amongst other things aspects of the ResMed Limited MIRAGE LIBERTY™ full-face mask); International Patent Application WO 2009/052,560 (describing amongst other things aspects of the ResMed Limited SWIFT™ FX nasal pillows).

Many of the seal forming structures of the prior art comprise an element made from silicone (or another similar polymer) which creates a seal against the patient's face. However, some patients may dislike the surface texture of silicone and/or its lack of breathability.

### 1.2.3.1.2 Positioning and Stabilising

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face.

One technique is the use of adhesives. See for example US Patent Application Publication No. US 2010/0000534. However, the use of adhesives may be uncomfortable for some.

Another technique is the use of one or more straps and/or stabilising harnesses. Many such harnesses suffer from being one or more of ill-fitting, bulky, uncomfortable and awkward to use.

### 1.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

### 1.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 1.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition in cooler climates, warm air applied generally to the face area in and about the patient interface is more comfortable than cold air. Humidifiers therefore often have the capacity to heat the flow of air was well as humidifying it.

### 1.2.3.5 Oxygen Source

Experts in this field have recognized that exercise for respiratory failure patients provides long term benefits that slow the progression of the disease, improve quality of life and extend patient longevity. Most stationary forms of exercise like tread mills and stationary bicycles, however, are too strenuous for these patients. As a result, the need for mobility has long been recognized. Until recently, this mobility has been facilitated by the use of small compressed oxygen tanks or cylinders mounted on a cart with dolly wheels. The disadvantage of these tanks is that they contain a finite amount of oxygen and are heavy, weighing about 50 pounds when mounted.

Oxygen concentrators have been in use for about 50 years to supply oxygen for respiratory therapy. Traditional oxygen concentrators have been bulky and heavy making ordinary ambulatory activities with them difficult and impractical. Recently, companies that manufacture large stationary oxygen concentrators began developing portable oxygen concentrators (POCs). The advantage of POCs is that they can produce a theoretically endless supply of oxygen. In order to make these devices small for mobility, the various systems necessary for the production of oxygen enriched gas are condensed. POCs seek to utilize their produced oxygen as efficiently as possible, in order to minimise weight, size, and power consumption. This may be achieved by delivering the oxygen as series of pulses or “boli”, each bolus timed to coincide with the start of inspiration. This therapy mode is known as pulsed or demand (oxygen) delivery (POD), in contrast with traditional continuous flow delivery more suited to stationary oxygen concentrators.

### 1.2.3.6 Data Management

There may be clinical reasons to obtain data to determine whether the patient prescribed with respiratory therapy has been “compliant”, e.g. that the patient has used their RPT device according to one or more “compliance rules”. One example of a compliance rule for CPAP therapy is that a patient, in order to be deemed compliant, is required to use the RPT device for at least four hours a night for at least 21 of 30 consecutive days. In order to determine a patient's compliance, a provider of the RPT device, such as a health care provider, may manually obtain data describing the patient's therapy using the RPT device, calculate the usage over a predetermined time period, and compare with the compliance rule. Once the health care provider has determined that the patient has used their RPT device according to the compliance rule, the health care provider may notify a third party that the patient is compliant.

There may be other aspects of a patient's therapy that would benefit from communication of therapy data to a third party or external system.

Existing processes to communicate and manage such data can be one or more of costly, time-consuming, and error-prone.

### 1.2.3.7 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

The vent may comprise an orifice and gas may flow through the orifice in use of the mask. Many such vents are noisy. Others may become blocked in use and thus provide insufficient washout. Some vents may be disruptive of the sleep of a bed partner 1100 of the patient 1000, e.g. through noise or focused airflow.

ResMed Limited has developed a number of improved mask vent technologies. See International Patent Application Publication No. WO 1998/034,665; International Patent Application Publication No. WO 2000/078,381; U.S. Pat. No. 6,581,594; US Patent Application Publication No. US 2009/0050156; US Patent Application Publication No. 2009/0044808.

Table of noise of prior masks (ISO 17510-2:2007, 10 cmH₂O pressure at 1 m)

| | | A-weighted | A-weighted | |
|---|---|---|---|---|
| | | sound power | sound pres- | |
| | | level dB(A) | sure dB(A) | Year |
| Mask name | Mask type | (uncertainty) | (uncertainty) | (approx.) |
| Glue-on (*) | nasal | 50.9 | 42.9 | 1981 |
| ResCare | nasal | 31.5 | 23.5 | 1993 |
| standard (*) | | | | |
| ResMed | nasal | 29.5 | 21.5 | 1998 |
| Mirage ™ (*) | | | | |
| ResMed | nasal | 36 (3) | 28 (3) | 2000 |
| UltraMirage ™ | | | | |
| ResMed | nasal | 32 (3) | 24 (3) | 2002 |
| Mirage | | | | |
| Activa ™ | | | | |
| ResMed | nasal | 30 (3) | 22 (3) | 2008 |
| Mirage | | | | |
| Micro ™ | | | | |
| ResMed | nasal | 29 (3) | 22 (3) | 2008 |
| Mirage ™ | | | | |
| SoftGel | | | | |
| ResMed | nasal | 26 (3) | 18 (3) | 2010 |
| Mirage ™ FX | | | | |
| ResMed | nasal pillows | 37 | 29 | 2004 |
| Mirage Swift ™ | | | | |
| (*) | | | | |
| ResMed | nasal pillows | 28 (3) | 20 (3) | 2005 |
| Mirage Swift ™ | | | | |
| II | | | | |
| ResMed | nasal pillows | 25 (3) | 17 (3) | 2008 |
| Mirage Swift ™ | | | | |
| LT | | | | |
| ResMed AirFit | nasal pillows | 21 (3) | 13 (3) | 2014 |
| P10 | | | | |

((*) one specimen only, measured using test method specified in ISO 3744 in CPAP mode at 10 cmH₂O)

Sound pressure values of a variety of objects are listed below

| | A-weighted sound | |
|---|---|---|
| Object | pressure dB(A) | Notes |
| Vacuum cleaner: Nilfisk | 68 | ISO 3744 at |
| Walter Broadly Litter | | 1 m distance |
| Hog: B+ Grade | | |
| Conversational speech | 60 | 1 m distance |
| Average home | 50 | |
| Quiet library | 40 | |
| Quiet bedroom at night | 30 | |
| Background in TV studio | 20 | |

### 1.2.4 Screening, Diagnosis, and Monitoring Systems

Polysomnography (PSG) is a conventional system for diagnosis and monitoring of cardio-pulmonary disorders, and typically involves expert clinical staff to apply the system. PSG typically involves the placement of 15 to 20 contact sensors on a patient in order to record various bodily signals such as electroencephalography (EEG), electrocardiography (ECG), electrooculograpy (EOG), electromyography (EMG), etc. PSG for sleep disordered breathing has involved two nights of observation of a patient in a clinic, one night of pure diagnosis and a second night of titration of treatment parameters by a clinician. PSG is therefore expensive and inconvenient. In particular it is unsuitable for home screening/diagnosis/monitoring of sleep disordered breathing.

Screening and diagnosis generally describe the identification of a condition from its signs and symptoms. Screening typically gives a true/false result indicating whether or not a patient's SDB is severe enough to warrant further investigation, while diagnosis may result in clinically actionable information. Screening and diagnosis tend to be one-off processes, whereas monitoring the progress of a condition can continue indefinitely. Some screening/diagnosis systems are suitable only for screening/diagnosis, whereas some may also be used for monitoring.

Clinical experts may be able to screen, diagnose, or monitor patients adequately based on visual observation of PSG signals. However, there are circumstances where a clinical expert may not be available, or a clinical expert may not be affordable. Different clinical experts may disagree on a patient's condition. In addition, a given clinical expert may apply a different standard at different times.

### 2 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

It is to be understood that where anatomical terms of location/direction (e.g. anterior, posterior, superior, inferior, lateral, medial etc.) are used in the description of apparatus or methods disclosed herein, the terms are to be understood to refer to position or direction when an apparatus is in an in-use position/orientation. For example, a reference to the anterior of a patient interface is to be understood to be a reference to the anterior of the patient interface when the patient interface is in an in-use. As a further example, a posterior-facing surface of a seal-forming structure is a surface which, when the patient interface having the seal-forming structure is donned on the patient's head and in use, faces posteriorly.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

Another aspect of one form of the present technology is to provide a patient interface that can flex to accommodate patients having faces of varying widths.

Another aspect of one form of the present technology is to provide an oro-nasal patient interface that is lightweight.

Another aspect of one form of the present technology is to provide an oro-nasal patient interface that is comfortable and low cost.

Another aspect of the present technology comprises a cushion module for a patient interface, the cushion module comprising:
a plenum chamber pressurisable to a therapeutic pressure of at least 6 cmH₂O above ambient air pressure, the plenum chamber configured to receive a flow of air at the therapeutic pressure for breathing by a patient;
a seal-forming structure constructed and arranged to form a seal with a region of the patient's face surrounding an entrance to the patient's airways, the seal-forming structure having at least one hole therein such that the flow of air at the therapeutic pressure is delivered to an entrance to the patient's nares and the patient's mouth, the seal-forming structure constructed and arranged to maintain said therapeutic pressure in the plenum chamber throughout the patient's respiratory cycle in use,
a chassis portion and a membrane portion together at least partially forming the plenum chamber, the membrane portion being attached to the chassis portion and forming the seal-forming structure, the chassis portion and membrane portion being formed separately;
wherein the membrane portion is attached to the chassis portion in an inverted position in which the membrane portion is positioned on an anterior side of the chassis portion, the membrane portion then being inverted to an in-use position in which the membrane portion is positioned on a posterior side of the chassis portion.

In examples:
the membrane portion is attached to the chassis portion at or proximate a periphery of the chassis portion;
the membrane portion is formed at least partially from a textile material;
the membrane portion comprises an air-impermeable layer and a textile layer, the textile layer forming a patient-facing side of the membrane-portion;
the chassis portion is formed at least partially from a foam material;
the chassis portion comprises an air-impermeable layer provided to the foam material on at least one of the anterior side and the posterior side of the chassis portion;
the air-impermeable layer of the chassis portion comprises a TPU film;
the chassis portion comprises a textile layer on at least one of the anterior side and the posterior side of the chassis portion; and/or
the chassis portion comprises a stiffening layer on an anterior side of the chassis portion.

In further examples:
the chassis portion comprises a nasal recess formed in the chassis portion and located proximate a pronasale region of the patient's nose in use, the nasal recess being recessed with respect to adjacent lateral portions of the chassis portion;
the nasal recess comprises a saddle portion formed in the chassis portion;
the membrane portion has a shape at the nasal recess which at least partially follows the shape of the chassis portion;
the membrane portion is slack at the nasal recess;
the cushion module is constructed and arranged to allow a pronasale region of the user's nose to lie in the nasal recess in use;
the cushion module is constructed and arranged to allow the pronasale of a patient with a long nose to protrude anteriorly of the anterior side of the chassis portion;
the membrane portion is taut at the nasal recess;
the membrane portion spans across the nasal recess from one lateral side of the nasal recess to the other lateral side of the nasal recess leaving space between the membrane portion and the chassis portion at the nasal recess;
the membrane portion is constructed and arranged to be deformed into or towards the shape of the nasal recess by the patient's nose in use;
the membrane portion comprises a first hole through which air can flow to both the patient's nares, in use; and/or
the membrane portion comprises a second hole through which air can flow to the patient's mouth, in use.

In further examples:
the chassis portion is formed by thermoforming;
the chassis portion is formed by injection moulding;
the membrane portion is attached to the chassis portion while held in a predetermined shape by vacuum during attachment to the chassis portion;
the membrane portion is attached to the chassis portion while under tension;
the membrane portion is stretched in a nasal region of the membrane portion while held in the predetermined shape during attachment such that the nasal region of the membrane portion is slack when in the in-use position;
the membrane portion is welded to the chassis portion; and/or
the membrane portion is attached to the chassis portion by one of ultrasonic welding, RF welding or laser welding.

In further examples:
the cushion module comprises headgear connection portions connected to the chassis portion, the headgear connection portions constructed and arranged to connect to a positioning and stabilising structure of the patient interface;
the headgear connection portions comprise a pair of superior headgear connection portions constructed and arranged to connect to superior strap portions of the positioning and stabilising structure of the patient interface; and/or
the headgear connection portions comprise a pair of inferior headgear connection portions constructed and arranged to connect to inferior strap portions of the positioning and stabilising structure of the patient interface.

Another form of the present technology comprises a patient interface comprising:
the cushion module according to the above aspect or any one of the subsequent examples;
a vent to allow a continuous flow of gases exhaled by the patient from an interior of the plenum chamber of the cushion module to ambient, said vent being sized and shaped to maintain the therapeutic pressure in the plenum chamber in use;
a positioning and stabilising structure a positioning and stabilising structure configured to provide a force to hold the seal-forming structure in a therapeutically effective position on the patient's head in use;
wherein the patient interface is configured to allow the patient to breathe from ambient through their mouth in the absence of a flow of pressurised air through the plenum chamber inlet port, or the patient interface is configured to leave the patient's mouth uncovered in use.

Another form of the present technology comprises a method of manufacturing a cushion module of a patient interface, the method comprising:
forming a chassis portion of the cushion module and a membrane portion of the cushion module separately, the chassis portion and the membrane portion constructed and arranged to together at least partially form a plenum chamber of the patient interface, the plenum chamber being pressurisable to a therapeutic pressure of at least 6 cmH₂O above ambient air pressure and being configured to receive a flow of air at the therapeutic pressure for breathing by a patient;
attaching the membrane portion to the chassis portion in an inverted position in which the membrane portion is positioned on an anterior side of the chassis portion;
inverting the membrane portion to an in-use position in which the membrane portion is positioned on a posterior side of the chassis portion;
wherein in the in-use position the membrane portion forms a seal-forming structure of the patient interface, the seal-forming structure constructed and arranged to form a seal with a region of the patient's face surrounding an entrance to the patient's airways, the at least one hole of the membrane portion being constructed and arranged to deliver the flow of air at the therapeutic pressure to an entrance to the patient's nares and the patient's mouth, the seal-forming structure constructed and arranged to maintain said therapeutic pressure in the plenum chamber throughout the patient's respiratory cycle in use.

In examples:
the step of forming the chassis portion comprises forming the chassis portion at least partially from a foam material;
the step of forming the chassis portion comprises thermoforming the chassis portion;
the step of forming the membrane portion comprises forming the membrane portion at least partially from a textile material;
the step of forming the membrane portion comprises forming the membrane portion from an elastomeric material;
the step of attaching the membrane portion to the chassis portion comprises attaching the membrane portion to the chassis portion at or proximate a periphery of the chassis portion;
the step of attaching the membrane portion to the chassis portion comprises holding the membrane portion in a predetermined shape by vacuum during attachment to the chassis portion;
the step of attaching the membrane portion to the chassis portion comprises tensioning at least a portion of the membrane portion during attachment;
the step of attaching the membrane portion to the chassis portion comprises stretching the membrane portion in a nasal region of the membrane portion while held in the predetermined shape during attachment such that the nasal region of the membrane portion is slack when in the in-use position;
the step of attaching the membrane portion to the chassis portion may comprise welding the membrane portion to the chassis portion; and/or
the step of attaching the membrane portion to the chassis portion may comprise one of RF welding, ultrasonic welding or laser welding the membrane portion to the chassis portion.

Another aspect of the present technology comprises a cushion module for a patient interface, the cushion module comprising:
a plenum chamber pressurisable to a therapeutic pressure of at least 6 cmH₂O above ambient air pressure, the plenum chamber configured to receive a flow of air at the therapeutic pressure for breathing by a patient;
a seal-forming structure constructed and arranged to form a seal with a region of the patient's face surrounding an entrance to the patient's airways, the seal-forming structure having at least one hole therein such that the flow of air at the therapeutic pressure is delivered to an entrance to the patient's nares and the patient's mouth, the seal-forming structure constructed and arranged to maintain said therapeutic pressure in the plenum chamber throughout the patient's respiratory cycle in use,
a chassis portion and a membrane portion together at least partially forming the plenum chamber, the membrane portion being attached to the chassis portion and forming the seal-forming structure;
wherein the membrane portion is formed at least partially from a textile material and the chassis portion is formed at least partially from a foam material and an air-impermeable layer provided to the foam material on at least one of an anterior side and a posterior side of the chassis portion, the chassis portion being formed by thermoforming.

In examples:
the membrane portion is attached to the chassis portion at or proximate a periphery of the chassis portion;
the membrane portion comprises an air-impermeable layer and a textile layer, the textile layer forming a patient-facing side of the membrane portion;
the air impermeable layer of the chassis portion is provided on both the anterior side and the posterior side of the chassis portion;
the air-impermeable layer of the chassis portion comprises a TPU film;
the chassis portion comprises a textile layer on at least one of the anterior side and the posterior side of the chassis portion;
the textile layer of the chassis portion is provided on both the anterior side and the posterior side of the chassis portion;
the chassis portion comprises a stiffening layer on an anterior side of the chassis portion;
the membrane portion is welded to the chassis portion; and/or
the membrane portion is attached to the chassis portion by one of ultrasonic welding, RF welding or laser welding.

In further examples:
the chassis portion comprises a nasal recess formed in the chassis portion and located proximate a pronasale region of the patient's nose in use, the nasal recess being recessed with respect to adjacent lateral portions of the chassis portion;
the nasal recess comprises a saddle portion formed in the chassis portion;
the membrane portion has a shape at the nasal recess which at least partially follows the shape of the chassis portion;
the membrane portion is slack at the nasal recess;
the cushion module is constructed and arranged to allow a pronasale region of the user's nose to lie in the nasal recess in use;
the cushion module is constructed and arranged to allow the pronasale of a patient with a long nose to protrude anteriorly of the anterior side of the chassis portion;
the membrane portion is taut at the nasal recess;
the membrane portion spans across the nasal recess from one lateral side of the nasal recess to the other lateral side of the nasal recess leaving space between the membrane portion and the chassis portion at the nasal recess; and/or
the membrane portion is constructed and arranged to be deformed into or towards the shape of the nasal recess by the patient's nose in use.

Another form of the present technology comprises a patient interface comprising:
the cushion module according to the above aspect or any one of the subsequent examples;
a vent to allow a continuous flow of gases exhaled by the patient from an interior of the plenum chamber of the cushion module to ambient, said vent being sized and shaped to maintain the therapeutic pressure in the plenum chamber in use;
a positioning and stabilising structure a positioning and stabilising structure configured to provide a force to hold the seal-forming structure in a therapeutically effective position on the patient's head in use;
wherein the patient interface is configured to allow the patient to breathe from ambient through their mouth in the absence of a flow of pressurised air through the plenum chamber inlet port, or the patient interface is configured to leave the patient's mouth uncovered in use.

Another form of the present technology comprises a cushion module for a patient interface comprising:
a plenum chamber pressurisable to a therapeutic pressure of at least 6 cmH₂O above ambient air pressure, the plenum chamber configured to receive a flow of air at the therapeutic pressure for breathing by a patient;
a seal-forming structure constructed and arranged to form a seal with a region of the patient's face surrounding an entrance to the patient's airways, the seal-forming structure having at least one hole therein such that the flow of air at the therapeutic pressure is delivered to an entrance to the patient's nares and the patient's mouth, the seal-forming structure constructed and arranged to maintain said therapeutic pressure in the plenum chamber throughout the patient's respiratory cycle in use,
a chassis portion and a membrane portion together at least partially forming the plenum chamber, the membrane portion being attached to the chassis portion and forming the seal-forming structure;
wherein the chassis portion is formed from a foam material and a covering provided to at least one portion of the foam material, the chassis portion being formed by injection moulding of the foam material against the covering.

In examples:
the covering is provided on two opposite sides of the chassis portion;
the covering comprises an anterior layer on an anterior side of the chassis portion and a posterior layer on a posterior side of the chassis portion;
the chassis portion is formed by injection moulding of the foam material between the anterior layer and the posterior layer;
the anterior layer and the posterior layer are integrally formed or are attached to each other; and/or
one or both of the anterior and posterior layers comprises a textile layer and/or an air-impermeable layer.

In further examples:
the membrane portion and the chassis portion are formed separately and the membrane portion is attached to the chassis portion at or proximate a periphery of the chassis portion;
the membrane portion is attached to a periphery of the chassis portion while the membrane portion is under tension;
the membrane portion is attached to the chassis portion in an inverted position in which the membrane portion is positioned on an anterior side of the chassis portion, the membrane portion then being inverted to an in-use position in which the membrane portion is positioned on a posterior side of the chassis portion;
the membrane portion is attached to the chassis portion by one of RF welding, ultrasonic welding and laser welding; and/or
the membrane portion is formed at least partially from a textile material.

In further examples:
the membrane portion is adhered to the chassis portion;
the chassis portion may comprise a flat portion having a shape and size corresponding to the shape and size of a periphery of the membrane portion, the periphery of the membrane portion being adhered to the flat portion;
the membrane portion is adhered to the chassis portion with a thermoset material; and/or
the thermoset material is silicone.

In further examples:
the chassis portion comprises a nasal recess formed in the chassis portion and located proximate a pronasale region of the patient's nose in use, the nasal recess being recessed with respect to adjacent lateral portions of the chassis portion;
the nasal recess comprises a saddle portion formed in the chassis portion;
the membrane portion has a shape at the nasal recess which at least partially follows the shape of the chassis portion;
the membrane portion is slack at the nasal recess;
the cushion module is constructed and arranged to allow a pronasale region of the user's nose to lie in the nasal recess in use;
the cushion module is constructed and arranged to allow the pronasale of a patient with a long nose to protrude anteriorly of the anterior side of the chassis portion;
the membrane portion is taut at the nasal recess;
the membrane portion spans across the nasal recess from one lateral side of the nasal recess to the other lateral side of the nasal recess leaving space between the membrane portion and the chassis portion at the nasal recess;
the membrane portion is constructed and arranged to be deformed into or towards the shape of the nasal recess by the patient's nose in use;
the membrane portion comprises a first hole through which air can flow to both the patient's nares, in use; and/or
the membrane portion comprises a second hole through which air can flow to the patient's mouth, in use.

In further examples:
the cushion module comprises headgear connection portions connected to the chassis portion, the headgear connection portions constructed and arranged to connect to a positioning and stabilising structure of the patient interface;
the headgear connection portions comprise a pair of superior headgear connection portions constructed and arranged to connect to superior strap portions of the positioning and stabilising structure of the patient interface;
the headgear connection portions comprise a pair of inferior headgear connection portions constructed and arranged to connect to inferior strap portions of the positioning and stabilising structure of the patient interface.

Another form of the present technology comprises a cushion module for a patient interface comprising:
a plenum chamber pressurisable to a therapeutic pressure of at least 6 cmH₂O above ambient air pressure, the plenum chamber configured to receive a flow of air at the therapeutic pressure for breathing by a patient;
a seal-forming structure constructed and arranged to form a seal with a region of the patient's face surrounding an entrance to the patient's airways, the seal-forming structure having at least one hole therein such that the flow of air at the therapeutic pressure is delivered to an entrance to the patient's nares and the patient's mouth, the seal-forming structure constructed and arranged to maintain said therapeutic pressure in the plenum chamber throughout the patient's respiratory cycle in use,
a chassis portion and a membrane portion together at least partially forming the plenum chamber, the membrane portion being attached to the chassis portion and forming the seal-forming structure;
wherein the chassis portion is formed from a foam material and is formed by injection moulding of the foam material, the foam material being overmoulded to the membrane portion.

In examples:
the foam material is overmoulded to the membrane portion such that the membrane portion is attached to the chassis portion at or proximate a periphery of the chassis portion;
the membrane portion is inverted after attachment to the chassis portion to provide the seal-forming structure on a posterior side of the chassis portion;
the foam material is overmoulded to the membrane portion such that the membrane portion is attached to an inwardly facing surface on a posterior side of the chassis portion; and/or
the chassis portion may be attached to a frame on an anterior side of the chassis portion, the frame constructed and arranged to reinforce the chassis portion.

In further examples:
the membrane portion is formed at least partially from a textile material;
the membrane portion comprises an air-impermeable layer and a textile layer, the textile layer forming a patient-facing side of the membrane-portion;
the chassis portion comprises a nasal recess formed in the chassis portion and located proximate a pronasale region of the patient's nose in use, the nasal recess being recessed with respect to adjacent lateral portions of the chassis portion;
the nasal recess comprises a saddle portion formed in the chassis portion;
the membrane portion has a shape at the nasal recess which at least partially follows the shape of the chassis portion;
the membrane portion is slack at the nasal recess;
the cushion module is constructed and arranged to allow a pronasale region of the user's nose to lie in the nasal recess in use;
the cushion module is constructed and arranged to allow the pronasale of a patient with a long nose to protrude anteriorly of the anterior side of the chassis portion;
the membrane portion is taut at the nasal recess;
the membrane portion spans across the nasal recess from one lateral side of the nasal recess to the other lateral side of the nasal recess leaving space between the membrane portion and the chassis portion at the nasal recess;
the membrane portion is constructed and arranged to be deformed into or towards the shape of the nasal recess by the patient's nose in use;
the membrane portion is held in a predetermined shape by vacuum during overmoulding of the chassis portion to the membrane portion;
the membrane portion is stretched in a nasal region of the membrane portion while held in the predetermined shape during overmoulding such that the nasal region of the membrane portion is slack after overmoulding;
the membrane portion comprises a first hole through which air can flow to both the patient's nares, in use; and/or
the membrane portion comprises a second hole through which air can flow to the patient's mouth, in use.

In further examples:
the cushion module comprises headgear connection portions connected to the chassis portion, the headgear connection portions constructed and arranged to connect to a positioning and stabilising structure of the patient interface;
the headgear connection portions comprise a pair of superior headgear connection portions constructed and arranged to connect to superior strap portions of the positioning and stabilising structure of the patient interface;
the headgear connection portions comprise a pair of inferior headgear connection portions constructed and arranged to connect to inferior strap portions of the positioning and stabilising structure of the patient interface.

Another form of the present technology comprises a cushion module for a patient interface comprising:
a plenum chamber pressurisable to a therapeutic pressure of at least 6 cmH₂O above ambient air pressure, the plenum chamber configured to receive a flow of air at the therapeutic pressure for breathing by a patient;
a seal-forming structure constructed and arranged to form a seal with a region of the patient's face surrounding an entrance to the patient's airways, the seal-forming structure having at least one hole therein such that the flow of air at the therapeutic pressure is delivered to an entrance to the patient's nares and the patient's mouth, the seal-forming structure constructed and arranged to maintain said therapeutic pressure in the plenum chamber throughout the patient's respiratory cycle in use,
a chassis portion and a membrane portion together at least partially forming the plenum chamber, the membrane portion being attached to the chassis portion and forming the seal-forming structure;
wherein the chassis portion comprises a first portion and a second portion, the membrane portion being attached to the first portion, and the second portion comprising a foam material attached to the first portion.

In examples:
the second portion is formed by injection moulding of the foam material;
the foam material is overmoulded to the first portion;
the foam material is overmoulded to both an anterior side and a posterior side of the first portion;
the foam material at least partially encapsulates the first portion;
the first portion of the chassis portion is constructed and arranged to stiffen the chassis portion;
the first portion is stiffer than the foam material;
the first portion is semi-rigid;
the first portion is formed from TPE or TPU;
the membrane portion is at least partially formed from a textile material; and/or
the membrane portion comprises a textile layer forming a patient-facing side of the membrane-portion, and an air impermeable layer.

In further examples:
the membrane portion is attached to the chassis portion at or proximate a periphery of the chassis portion;
the chassis portion comprises a nasal recess formed in the chassis portion and located proximate a pronasale region of the patient's nose in use, the nasal recess being recessed with respect to adjacent lateral portions of the chassis portion;
the nasal recess comprises a saddle portion formed in the chassis portion;
the membrane portion has a shape at the nasal recess which at least partially follows the shape of the chassis portion;
the membrane portion is slack at the nasal recess;
the cushion module is constructed and arranged to allow a pronasale region of the user's nose to lie in the nasal recess in use;
the cushion module is constructed and arranged to allow the pronasale of a patient with a long nose to protrude anteriorly of the anterior side of the chassis portion;
the membrane portion is taut at the nasal recess;
the membrane portion spans across the nasal recess from one lateral side of the nasal recess to the other lateral side of the nasal recess leaving space between the membrane portion and the chassis portion at the nasal recess;
the membrane portion is constructed and arranged to be deformed into or towards the shape of the nasal recess by the patient's nose in use;
the membrane portion comprises a first hole through which air can flow to both the patient's nares, in use; and/or
the membrane portion comprises a second hole through which air can flow to the patient's mouth, in use.

In further examples:
the cushion module comprises headgear connection portions connected to the chassis portion, the headgear connection portions constructed and arranged to connect to a positioning and stabilising structure of the patient interface;
the headgear connection portions comprise a pair of superior headgear connection portions constructed and arranged to connect to superior strap portions of the positioning and stabilising structure of the patient interface;
the headgear connection portions comprise a pair of inferior headgear connection portions constructed and arranged to connect to inferior strap portions of the positioning and stabilising structure of the patient interface.

Another form of the present technology comprises a cushion module for a patient interface comprising:
a plenum chamber pressurisable to a therapeutic pressure of at least 6 cmH₂O above ambient air pressure, the plenum chamber configured to receive a flow of air at the therapeutic pressure for breathing by a patient;
a seal-forming structure constructed and arranged to form a seal with a region of the patient's face surrounding an entrance to the patient's airways, the seal-forming structure having at least one hole therein such that the flow of air at the therapeutic pressure is delivered to an entrance to the patient's nares and the patient's mouth, the seal-forming structure constructed and arranged to maintain said therapeutic pressure in the plenum chamber throughout the patient's respiratory cycle in use,
a chassis portion and a membrane portion together at least partially forming the plenum chamber, the membrane portion being attached to the chassis portion and forming the seal-forming structure;
wherein the cushion module further comprises an insert configured to attach to or fit within a portion of the chassis portion.

In examples:
the insert is formed from a foam material;
the foam material is a silicone foam;
the foam material is a TPU foam;
the insert is insertable into an anterior side of the chassis portion;
the insert is configured to reinforce the chassis portion;
the insert is configured to function as an undercushion;
the insert is configured to support the membrane portion to seal between the patient's nasal ala and nasolabial sulci;
the insert is insertable into an interior of the plenum chamber; and/or
the insert is air-permeable and is configured to block the or each plenum chamber inlet port to diffuse the flow of air supplied to the plenum chamber.

In further examples:
the membrane portion is attached to the chassis portion at or proximate a periphery of the chassis portion;
the chassis portion is formed by thermoforming;
the chassis portion is formed by injection moulding;
the membrane portion is formed at least partially from a textile material;
the membrane portion comprises an air-impermeable layer and a textile layer, the textile layer forming a patient-facing side of the membrane-portion;
the chassis portion is formed at least partially from a foam material;
the chassis portion comprises an air-impermeable layer provided to the foam material of the chassis portion on at least one of the anterior side and the posterior side of the chassis portion;
the air-impermeable layer of the chassis portion comprises a TPU film;
the chassis portion comprises a textile layer on at least one of the anterior side and the posterior side of the chassis portion.

In further examples:
the chassis portion comprises a nasal recess formed in the chassis portion and located proximate a pronasale region of the patient's nose in use, the nasal recess being recessed with respect to adjacent lateral portions of the chassis portion;
the nasal recess comprises a saddle portion formed in the chassis portion;
the membrane portion has a shape at the nasal recess which at least partially follows the shape of the chassis portion;
the membrane portion is slack at the nasal recess;
the cushion module is constructed and arranged to allow a pronasale region of the user's nose to lie in the nasal recess in use;
the cushion module is constructed and arranged to allow the pronasale of a patient with a long nose to protrude anteriorly of the anterior side of the chassis portion;
the membrane portion is taut at the nasal recess;
the membrane portion spans across the nasal recess from one lateral side of the nasal recess to the other lateral side of the nasal recess leaving space between the membrane portion and the chassis portion at the nasal recess;
the membrane portion is constructed and arranged to be deformed into or towards the shape of the nasal recess by the patient's nose in use;
the membrane portion comprises a first hole through which air can flow to both the patient's nares, in use; and/or
the membrane portion comprises a second hole through which air can flow to the patient's mouth, in use.

In further examples:
the cushion module comprises headgear connection portions connected to the chassis portion, the headgear connection portions constructed and arranged to connect to a positioning and stabilising structure of the patient interface;
the headgear connection portions comprise a pair of superior headgear connection portions constructed and arranged to connect to superior strap portions of the positioning and stabilising structure of the patient interface;
the headgear connection portions comprise a pair of inferior headgear connection portions constructed and arranged to connect to inferior strap portions of the positioning and stabilising structure of the patient interface.

Another form of the present technology comprises a cushion module for a patient interface comprising:
a plenum chamber pressurisable to a therapeutic pressure of at least 6 cmH₂O above ambient air pressure, the plenum chamber configured to receive a flow of air at the therapeutic pressure for breathing by a patient;
a seal-forming structure constructed and arranged to form a seal with a region of the patient's face surrounding an entrance to the patient's airways, the seal-forming structure having at least one hole therein such that the flow of air at the therapeutic pressure is delivered to an entrance to the patient's nares and the patient's mouth, the seal-forming structure constructed and arranged to maintain said therapeutic pressure in the plenum chamber throughout the patient's respiratory cycle in use,
a vent to allow a continuous flow of gases exhaled by the patient from an interior of the plenum chamber to ambient, said vent being sized and shaped to maintain the therapeutic pressure in the plenum chamber in use;
wherein the patient interface is configured to allow the patient to breathe from ambient through their mouth in the absence of a flow of pressurised air through the plenum chamber inlet port, or the patient interface is configured to leave the patient's mouth uncovered in use;
wherein the patient interface comprises a chassis portion and a membrane portion together at least partially forming the plenum chamber, the membrane portion being attached to the chassis portion and forming the seal-forming structure;
wherein the chassis portion is at least partially formed from a material forming a heat and moisture exchange portion (HMX portion) functioning as a heat and moisture exchanger (HMX), the flow of air at the therapeutic pressure being provided through the HMX portion to enter the plenum chamber, and the vent positioned such that the flow of gases exhaled by the patient passed through the heat and moisture exchange portion prior to flowing through the vent.

In examples:
the chassis portion and HMX portion are integrally formed from foam;
the chassis portion comprises compression cut foam;
the chassis portion is formed by injection moulding of a foam material;
the membrane portion is attached to the chassis portion at or proximate a periphery of the chassis portion;
the membrane portion is formed at least partially from a textile material; and/or
the membrane portion comprises an air-impermeable layer and a textile layer, the textile layer forming a patient-facing side of the membrane-portion.

In further examples:
the chassis portion comprises a nasal recess formed in the chassis portion and located proximate a pronasale region of the patient's nose in use, the nasal recess being recessed with respect to adjacent lateral portions of the chassis portion;
the nasal recess comprises a saddle portion formed in the chassis portion;
the membrane portion has a shape at the nasal recess which at least partially follows the shape of the chassis portion;
the membrane portion is slack at the nasal recess;
the cushion module is constructed and arranged to allow a pronasale region of the user's nose to lie in the nasal recess in use;
the cushion module is constructed and arranged to allow the pronasale of a patient with a long nose to protrude anteriorly of the anterior side of the chassis portion;
the membrane portion is taut at the nasal recess;
the membrane portion spans across the nasal recess from one lateral side of the nasal recess to the other lateral side of the nasal recess leaving space between the membrane portion and the chassis portion at the nasal recess;
the membrane portion is constructed and arranged to be deformed into or towards the shape of the nasal recess by the patient's nose in use;
the membrane portion comprises a first hole through which air can flow to both the patient's nares, in use; and/or
the membrane portion comprises a second hole through which air can flow to the patient's mouth, in use.

In further examples:
the cushion module comprises headgear connection portions connected to the chassis portion, the headgear connection portions constructed and arranged to connect to a positioning and stabilising structure of the patient interface;
the headgear connection portions comprise a pair of superior headgear connection portions constructed and arranged to connect to superior strap portions of the positioning and stabilising structure of the patient interface;
the headgear connection portions comprise a pair of inferior headgear connection portions constructed and arranged to connect to inferior strap portions of the positioning and stabilising structure of the patient interface.

Another aspect of one form of the present technology is a patient interface that is moulded or otherwise constructed with a perimeter shape which is complementary to that of an intended wearer.

An aspect of one form of the present technology is a method of manufacturing apparatus.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a portable RPT device that may be carried by a person, e.g., around the home of the person.

An aspect of one form of the present technology is a patient interface that may be washed in a home of a patient, e.g., in soapy water, without requiring specialised cleaning equipment. An aspect of one form of the present technology is a humidifier tank that may be washed in a home of a patient, e.g., in soapy water, without requiring specialised cleaning equipment.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 3 BRIEF DESCRIPTION OF THE DRAWINGS

The present technology is illustrated by way of example, and not by way of limitation, in the figures of the accompanying drawings, in which like reference numerals refer to similar elements including:

### 3.1 Respiratory Therapy Systems

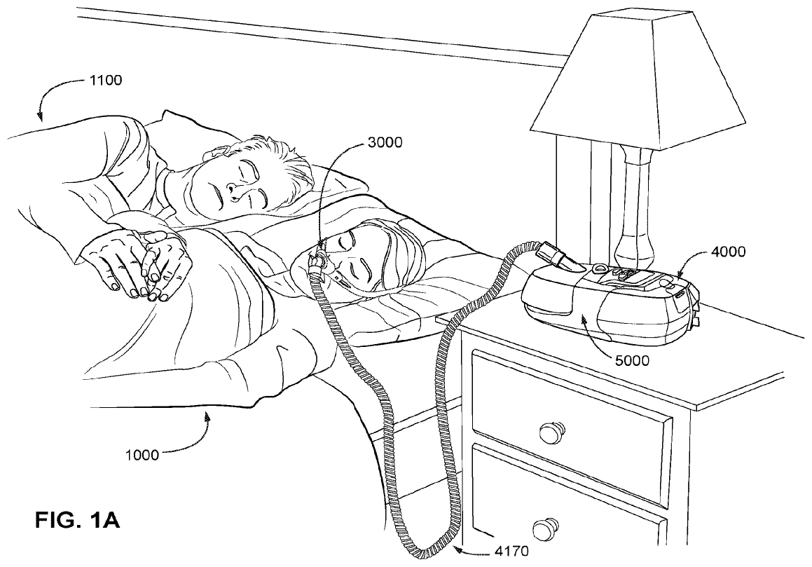

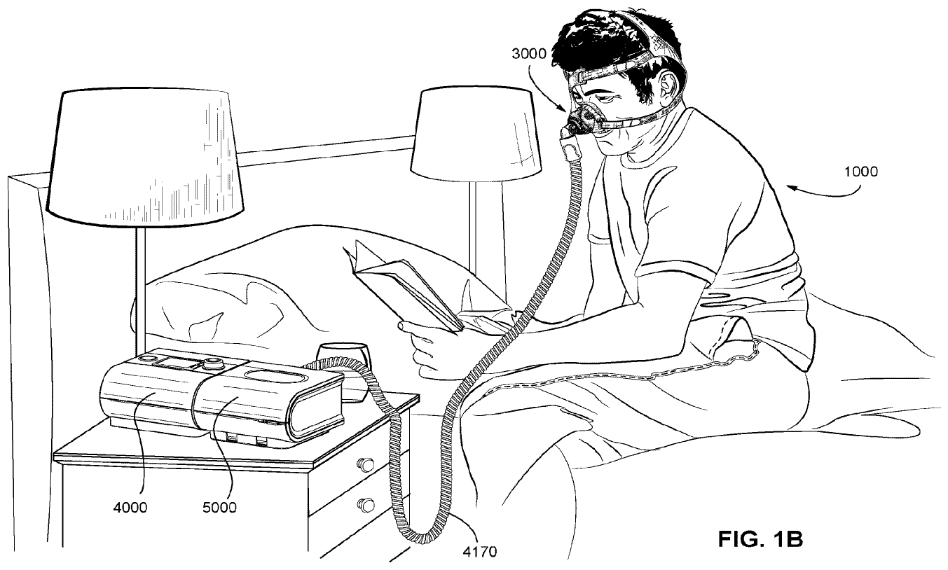

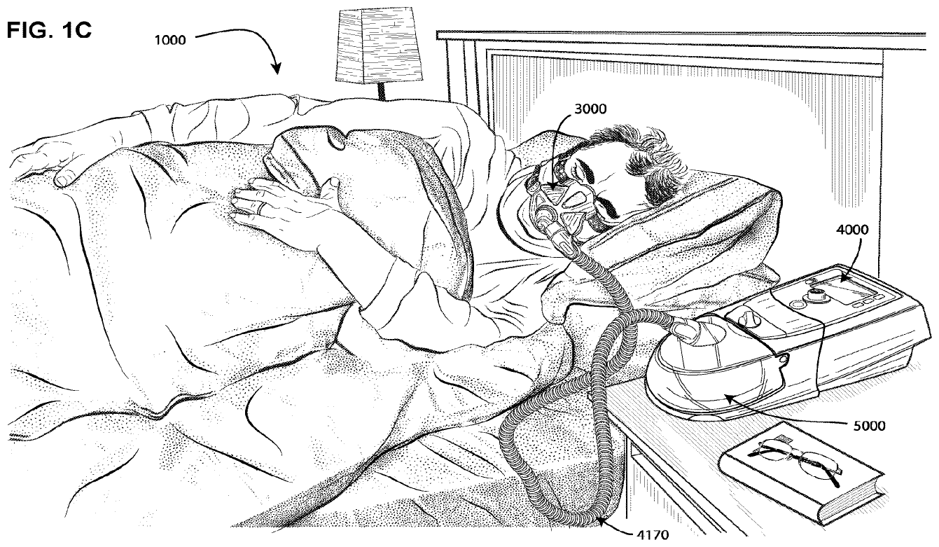

FIG. 1A shows a system including a patient 1000 wearing a patient interface 3000, in the form of nasal pillows, receiving a supply of air at positive pressure from an RPT device 4000. Air from the RPT device 4000 is conditioned in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 3.2 Respiratory System and Facial Anatomy

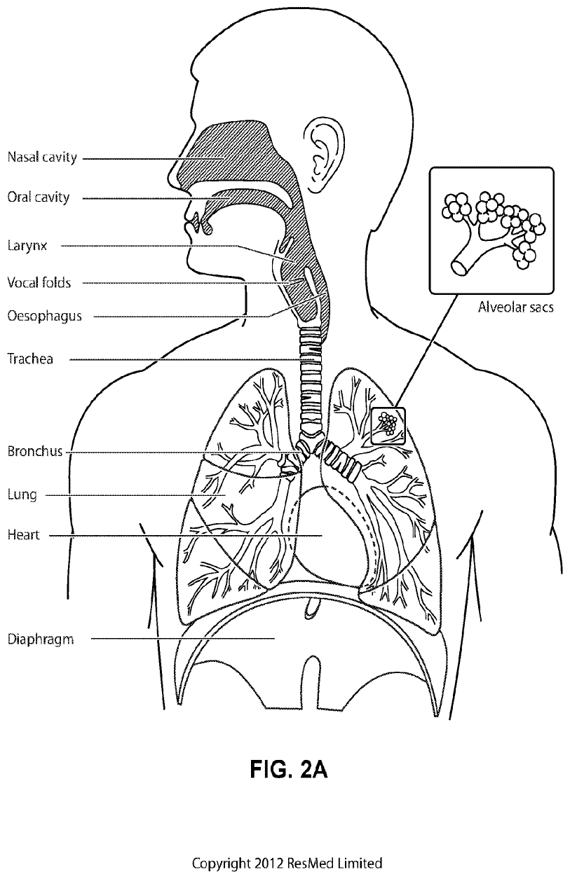

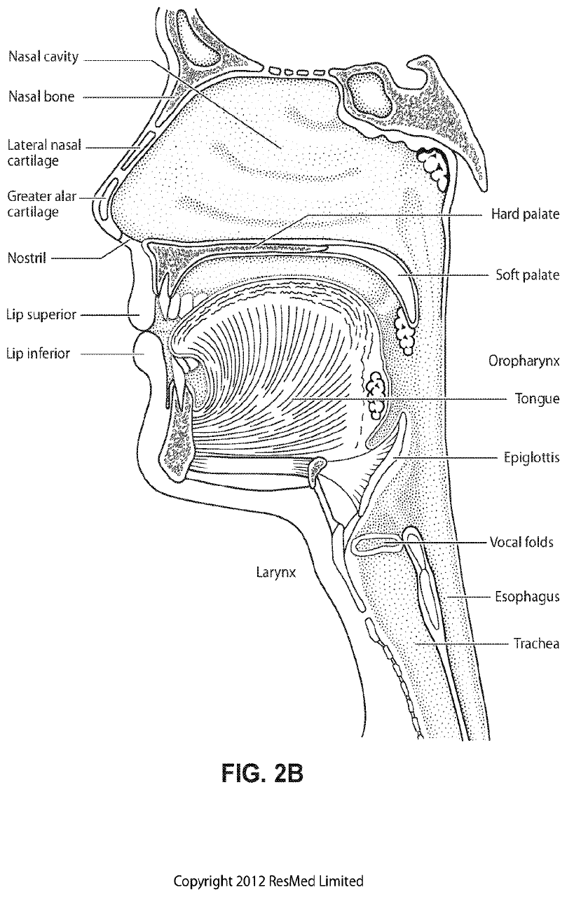

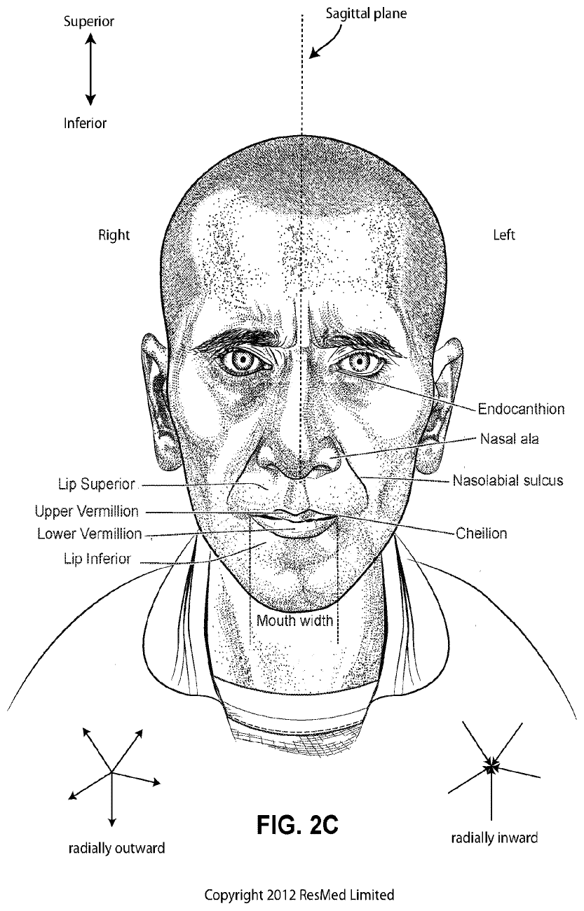

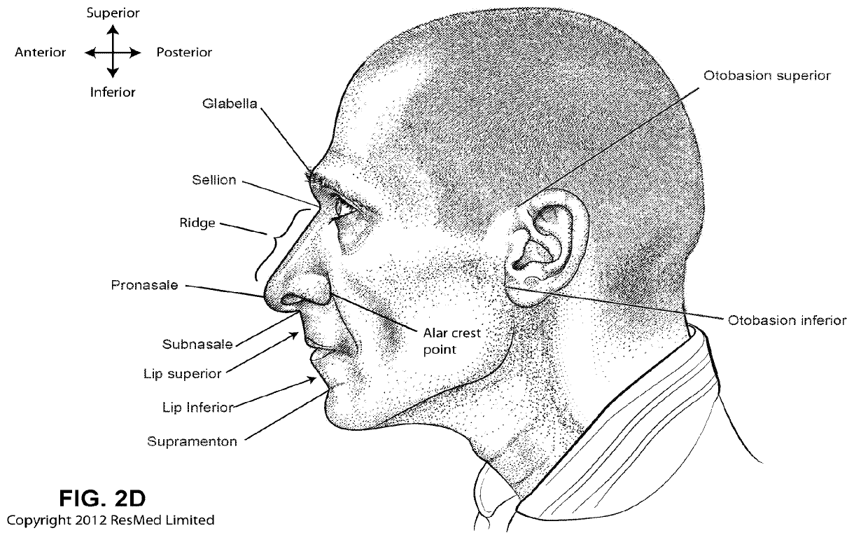

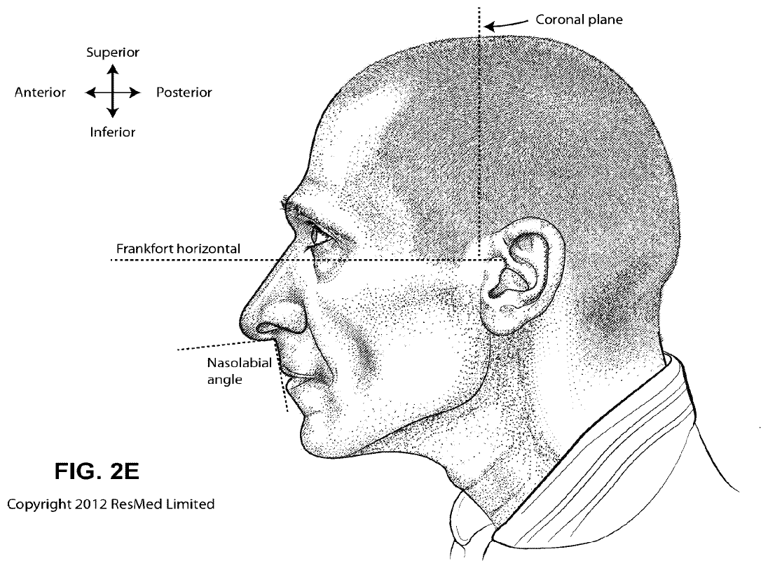

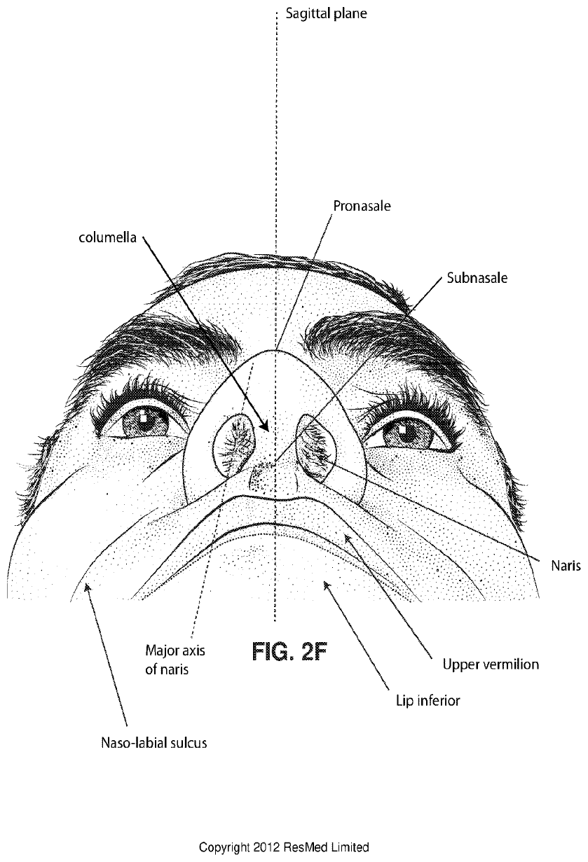

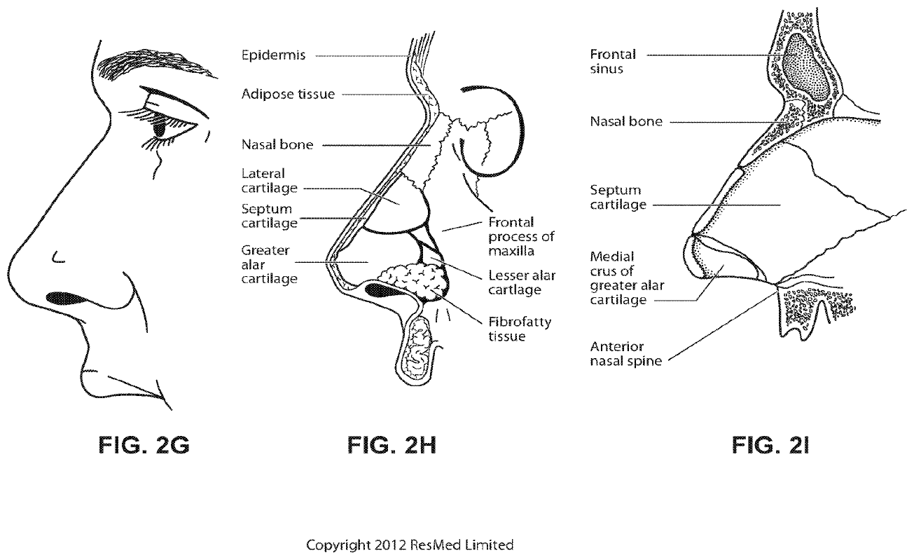

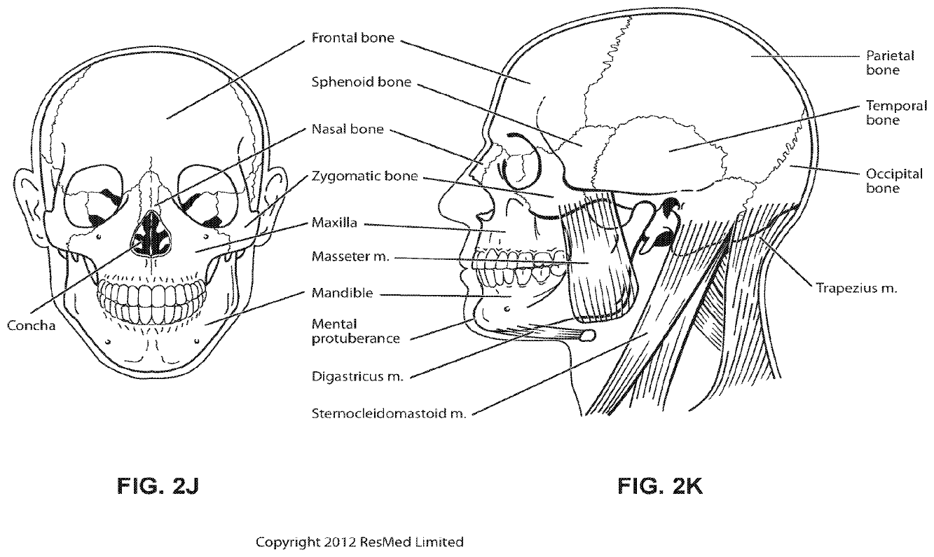

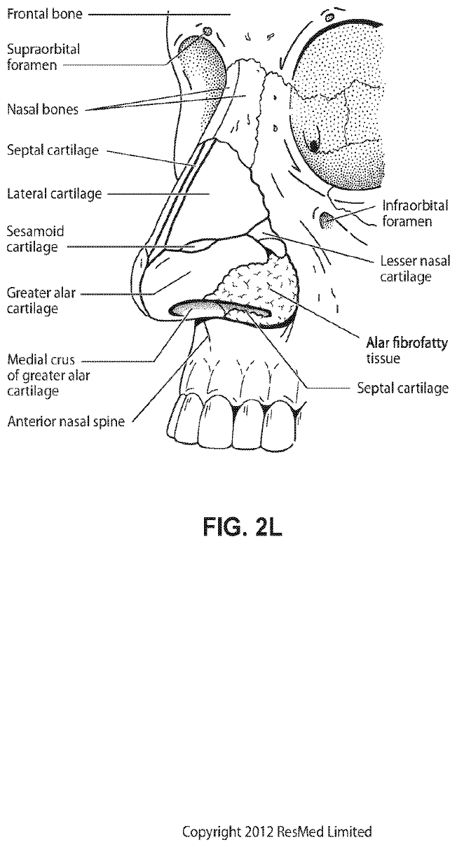

FIG. 2A shows an overview of a human respiratory system including the nasal and oral cavities, the larynx, vocal folds, oesophagus, trachea, bronchus, lung, alveolar sacs, heart and diaphragm.

FIG. 2B shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

FIG. 2C is a front view of a face with several features of surface anatomy identified including the lip superior, upper vermilion, lower vermilion, lip inferior, mouth width, endocanthion, a nasal ala, nasolabial sulcus and cheilion. Also indicated are the directions superior, inferior, radially inward and radially outward.

FIG. 2D is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2E is a further side view of a head. The approximate locations of the Frankfort horizontal and nasolabial angle are indicated. The coronal plane is also indicated.

FIG. 2F shows a base view of a nose with several features identified including naso-labial sulcus, lip inferior, upper Vermilion, naris, subnasale, columella, pronasale, the major axis of a naris and the midsagittal plane.

FIG. 2G shows a side view of the superficial features of a nose.

FIG. 2H shows subcutaneal structures of the nose, including lateral cartilage, septum cartilage, greater alar cartilage, lesser alar cartilage, sesamoid cartilage, nasal bone, epidermis, adipose tissue, frontal process of the maxilla and fibrofatty tissue.

FIG. 2I shows a medial dissection of a nose, approximately several millimeters from the midsagittal plane, amongst other things showing the septum cartilage and medial crus of greater alar cartilage.

FIG. 2J shows a front view of the bones of a skull including the frontal, nasal and zygomatic bones. Nasal concha are indicated, as are the maxilla, and mandible.

FIG. 2K shows a lateral view of a skull with the outline of the surface of a head, as well as several muscles. The following bones are shown: frontal, sphenoid, nasal, zygomatic, maxilla, mandible, parietal, temporal and occipital. The mental protuberance is indicated. The following muscles are shown: digastricus, masseter, sternocleidomastoid and trapezius.

FIG. 2L shows an anterolateral view of a nose.

### 3.3 Patient Interface

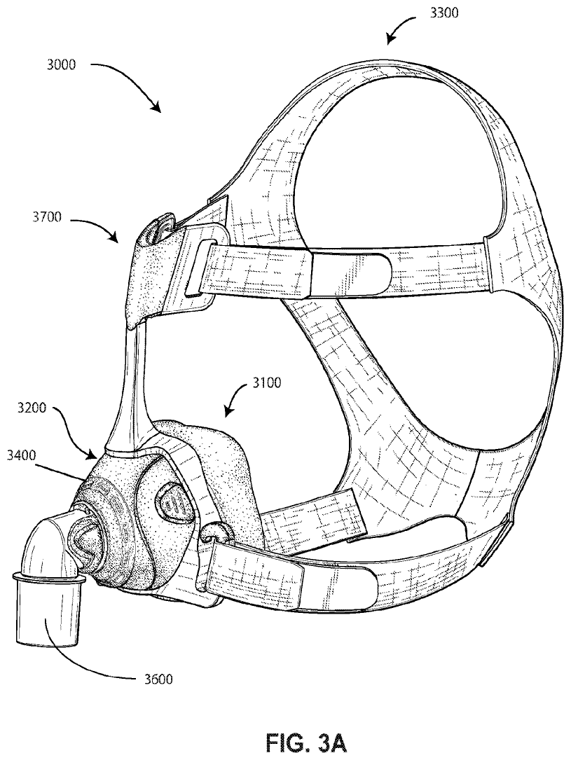

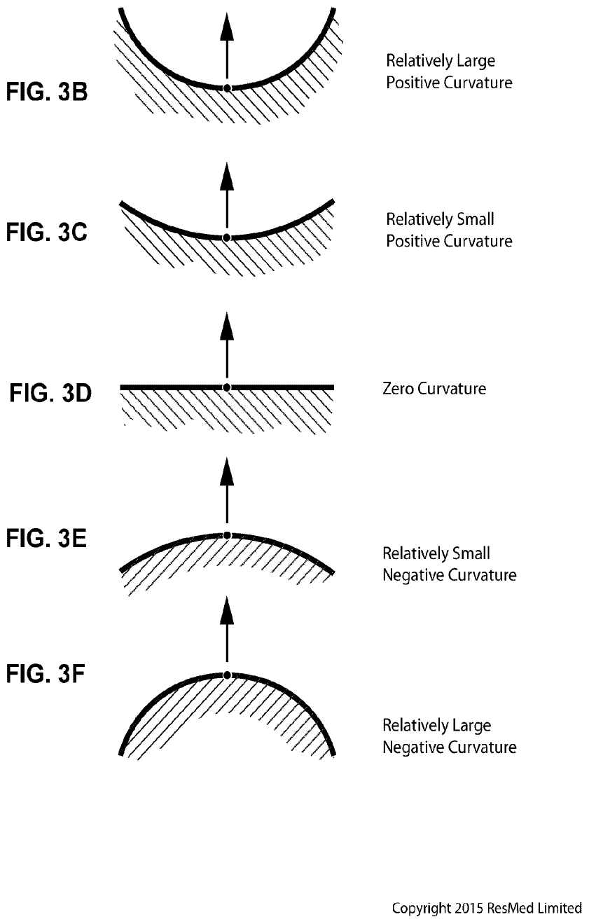

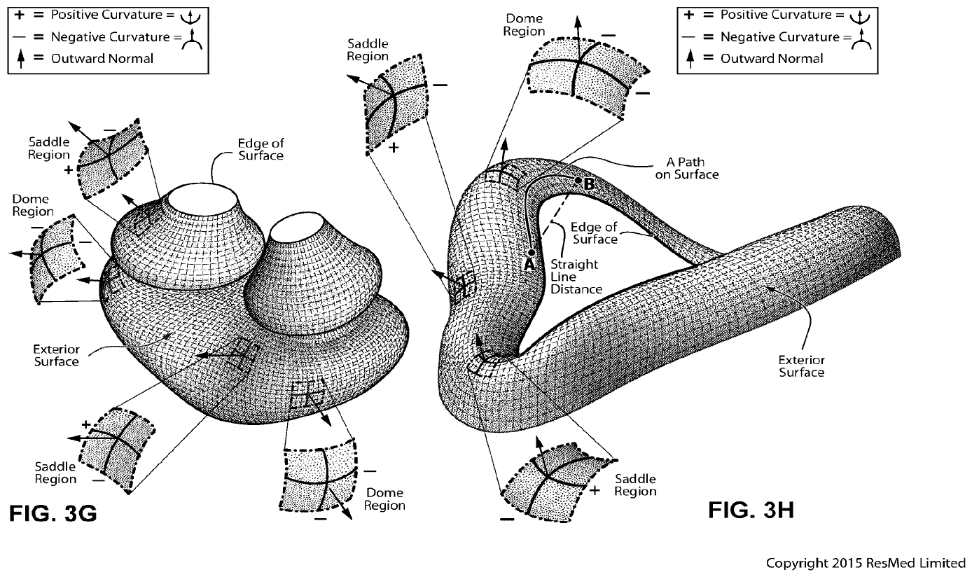

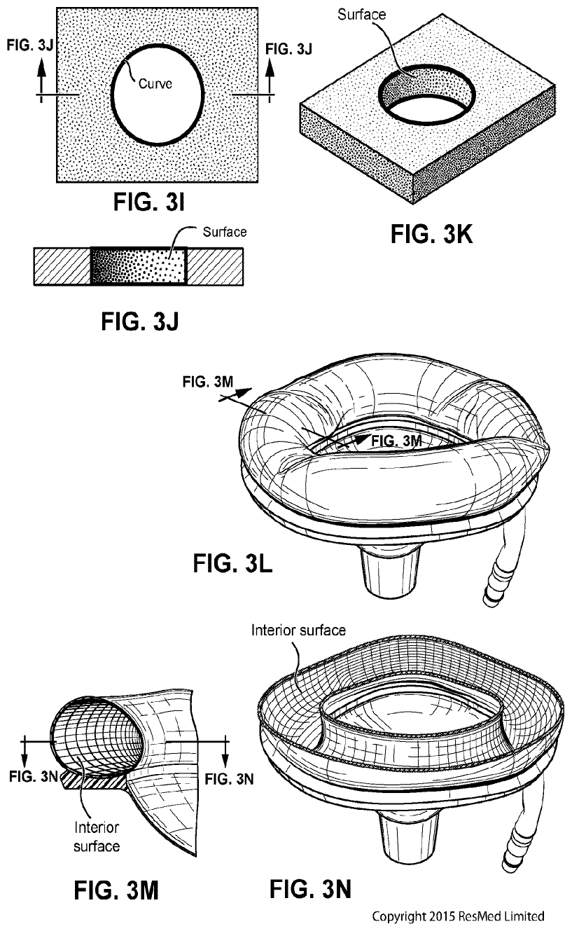

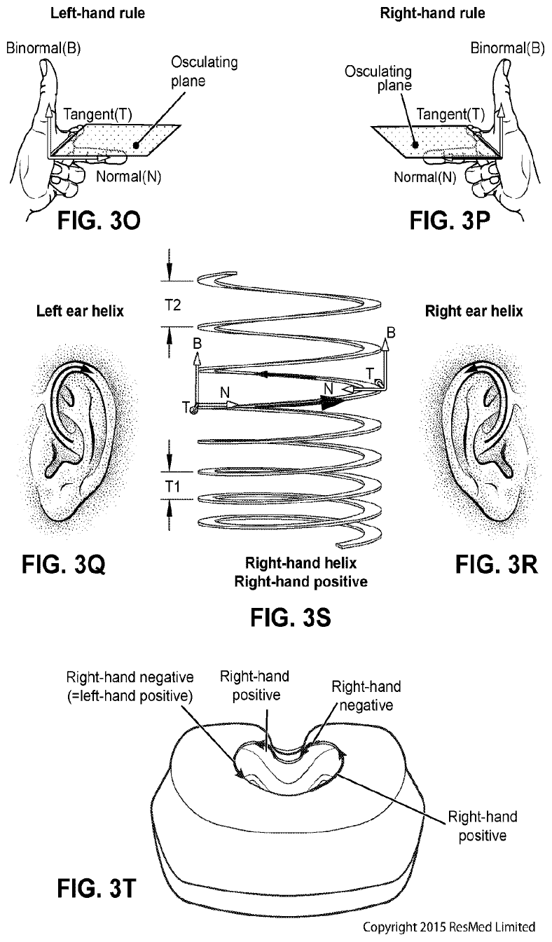

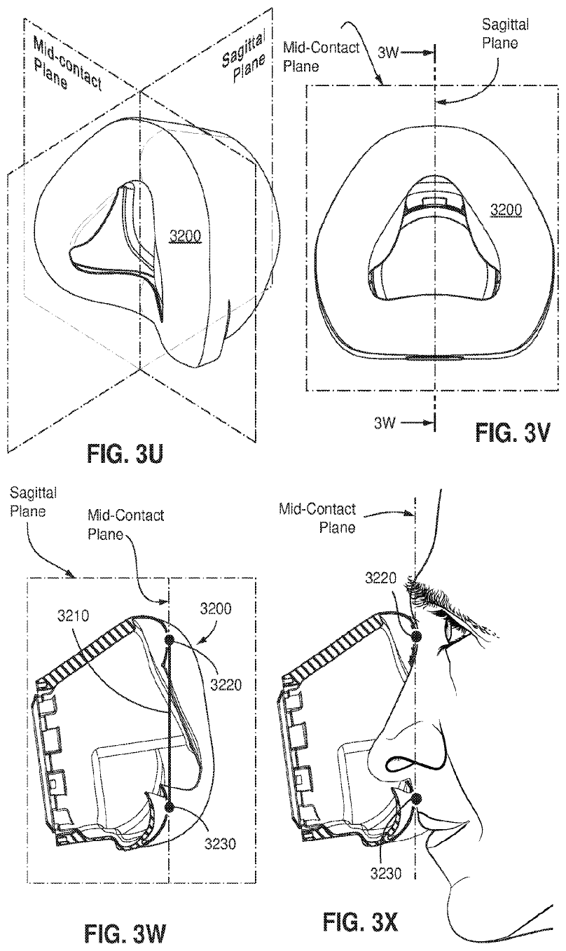

FIG. 3A shows a patient interface in the form of a nasal mask in accordance with one form of the present technology.

FIG. 3B shows a schematic of a cross-section through a structure at a point. An outward normal at the point is indicated. The curvature at the point has a positive sign, and a relatively large magnitude when compared to the magnitude of the curvature shown in FIG. 3C.

FIG. 3C shows a schematic of a cross-section through a structure at a point. An outward normal at the point is indicated. The curvature at the point has a positive sign, and a relatively small magnitude when compared to the magnitude of the curvature shown in FIG. 3B.

FIG. 3D shows a schematic of a cross-section through a structure at a point. An outward normal at the point is indicated. The curvature at the point has a value of zero.

FIG. 3E shows a schematic of a cross-section through a structure at a point. An outward normal at the point is indicated. The curvature at the point has a negative sign, and a relatively small magnitude when compared to the magnitude of the curvature shown in FIG. 3F.

FIG. 3F shows a schematic of a cross-section through a structure at a point. An outward normal at the point is indicated. The curvature at the point has a negative sign, and a relatively large magnitude when compared to the magnitude of the curvature shown in FIG. 3E.

FIG. 3G shows a cushion for a mask that includes two pillows. An exterior surface of the cushion is indicated. An edge of the surface is indicated. Dome and saddle regions are indicated.

FIG. 3H shows a cushion for a mask. An exterior surface of the cushion is indicated. An edge of the surface is indicated. A path on the surface between points A and B is indicated. A straight line distance between A and B is indicated. Two saddle regions and a dome region are indicated.

FIG. 3I shows the surface of a structure, with a one dimensional hole in the surface. The illustrated plane curve forms the boundary of a one dimensional hole.

FIG. 3J shows a cross-section through the structure of FIG. 3I. The illustrated surface bounds a two dimensional hole in the structure of FIG. 3I.

FIG. 3K shows a perspective view of the structure of FIG. 3I, including the two dimensional hole and the one dimensional hole. Also shown is the surface that bounds a two dimensional hole in the structure of FIG. 3I.

FIG. 3L shows a mask having an inflatable bladder as a cushion.

FIG. 3M shows a cross-section through the mask of FIG. 3L, and shows the interior surface of the bladder. The interior surface bounds the two dimensional hole in the mask.

FIG. 3N shows a further cross-section through the mask of FIG. 3L. The interior surface is also indicated.

FIG. 3O illustrates a left-hand rule.

FIG. 3P illustrates a right-hand rule.

FIG. 3Q shows a left ear, including the left ear helix.

FIG. 3R shows a right ear, including the right ear helix.

FIG. 3S shows a right-hand helix.

FIG. 3T shows a view of a mask, including the sign of the torsion of the space curve defined by the edge of the sealing membrane in different regions of the mask.

FIG. 3U shows a view of a plenum chamber 3200 showing a sagittal plane and a mid-contact plane.

FIG. 3V shows a view of a posterior of the plenum chamber of FIG. 3U. The direction of the view is normal to the mid-contact plane. The sagittal plane in FIG. 3V bisects the plenum chamber into left-hand and right-hand sides.

FIG. 3W shows a cross-section through the plenum chamber of FIG. 3V, the cross-section being taken at the sagittal plane shown in FIG. 3V. A ‘mid-contact’ plane is shown. The mid-contact plane is perpendicular to the sagittal plane. The orientation of the mid-contact plane corresponds to the orientation of a chord 3210 which lies on the sagittal plane and just touches the cushion of the plenum chamber at two points on the sagittal plane: a superior point 3220 and an inferior point 3230. Depending on the geometry of the cushion in this region, the mid-contact plane may be a tangent at both the superior and inferior points.

FIG. 3X shows the plenum chamber 3200 of FIG. 3U in position for use on a face. The sagittal plane of the plenum chamber 3200 generally coincides with the midsagittal plane of the face when the plenum chamber is in position for use. The mid-contact plane corresponds generally to the ‘plane of the face’ when the plenum chamber is in position for use. In FIG. 3X the plenum chamber 3200 is that of a nasal mask, and the superior point 3220 sits approximately on the sellion, while the inferior point 3230 sits on the lip superior.

FIG. 3Y shows a patient interface in the form of a nasal cannula in accordance with one form of the present technology.

### 3.4 RPT Device

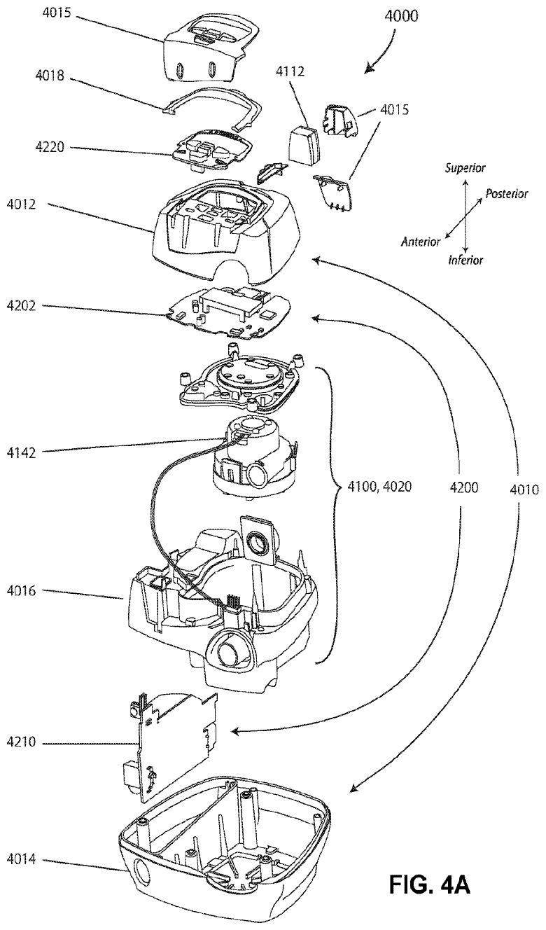

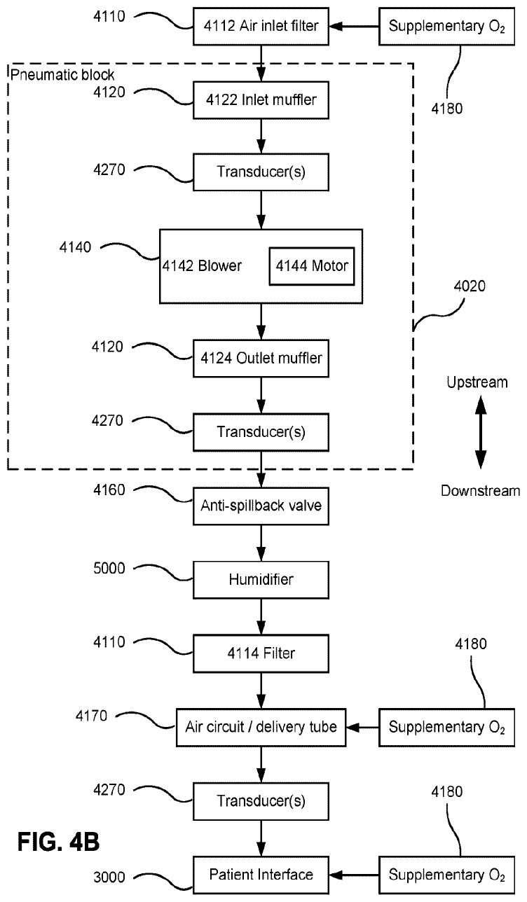

FIG. 4A shows an RPT device in accordance with one form of the present technology.

FIG. 4B is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

### 3.5 Humidifier

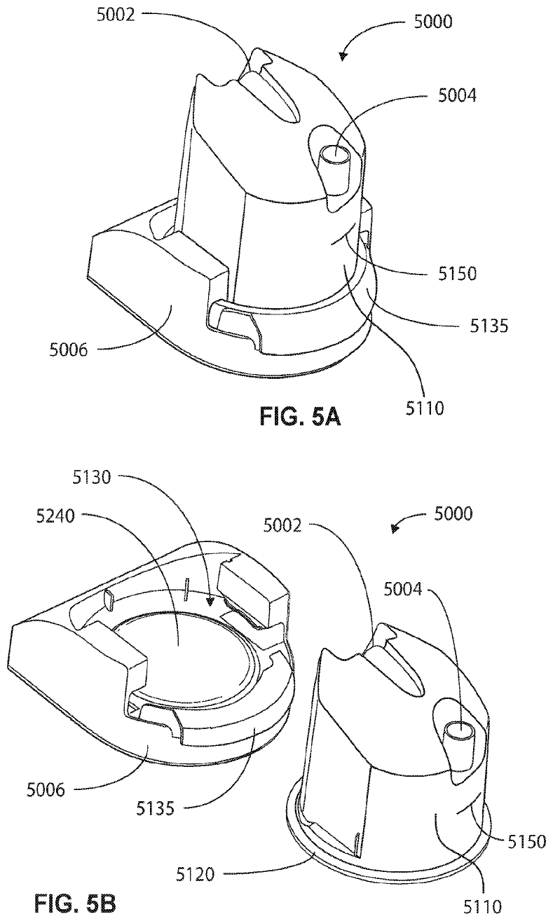

FIG. 5A shows an isometric view of a humidifier in accordance with one form of the present technology.

FIG. 5B shows an isometric view of a humidifier in accordance with one form of the present technology, showing a humidifier reservoir 5110 removed from the humidifier reservoir dock 5130.

### 3.6 Breathing Waveforms

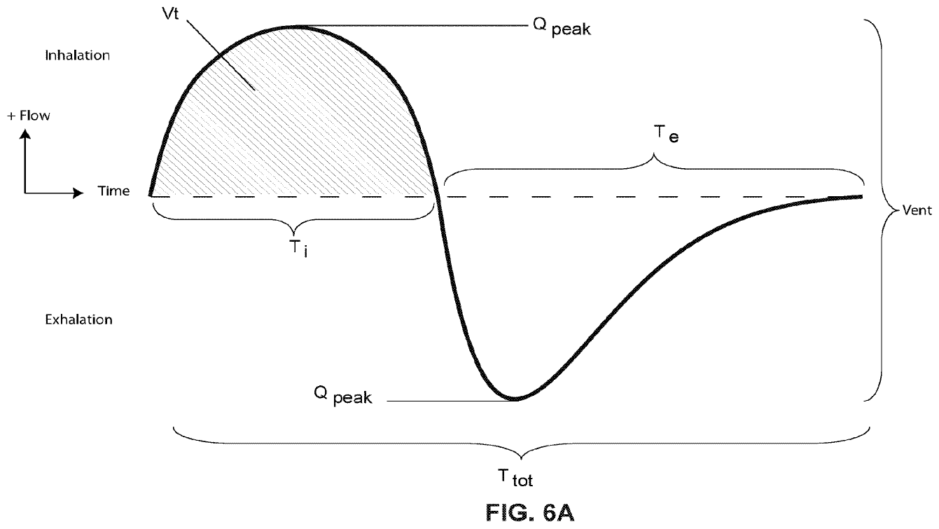

FIG. 6A shows a model typical breath waveform of a person while sleeping.

### 3.7 Patient Interfaces of the Present Technology

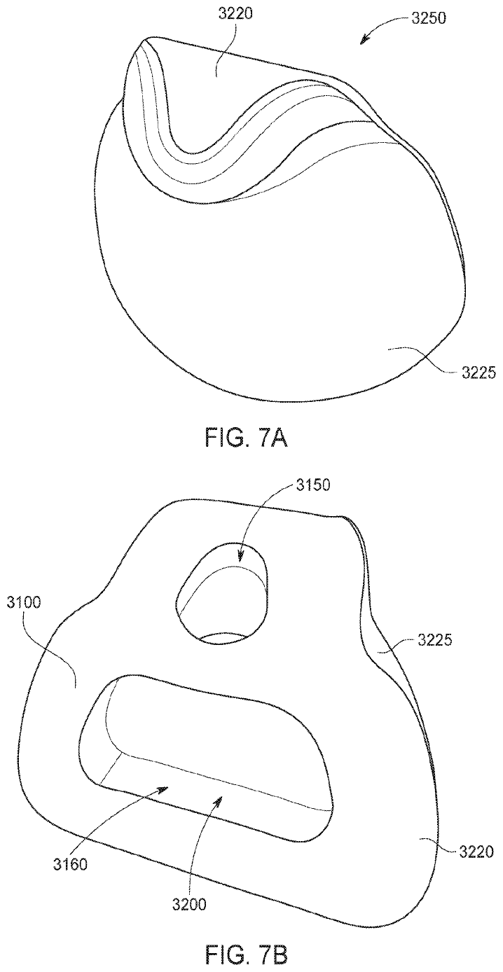

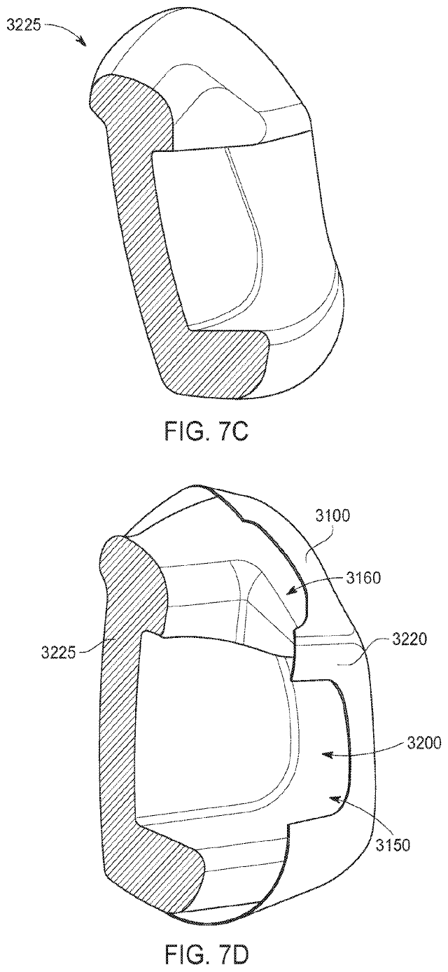

FIGS. 7A-7D show views of a cushion module of a patient interface according to one form of the present technology.

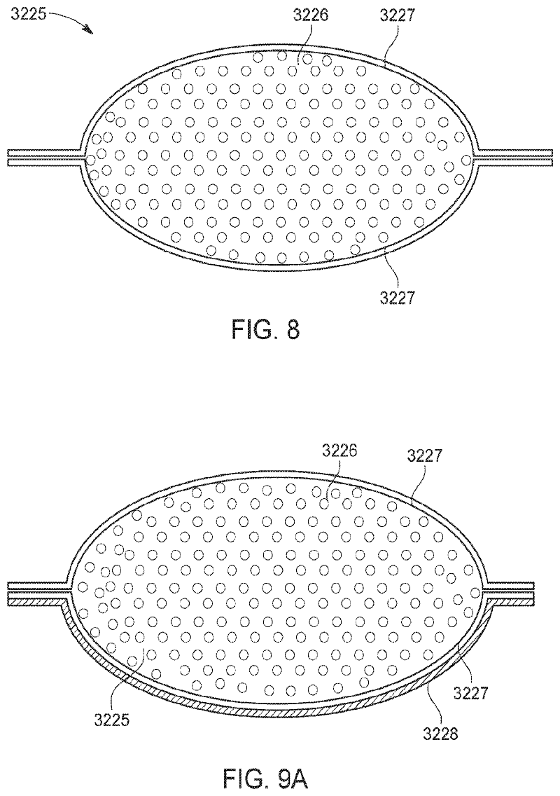

FIG. 8 shows a cross section schematic view of an undercushion according to one example of the present technology.

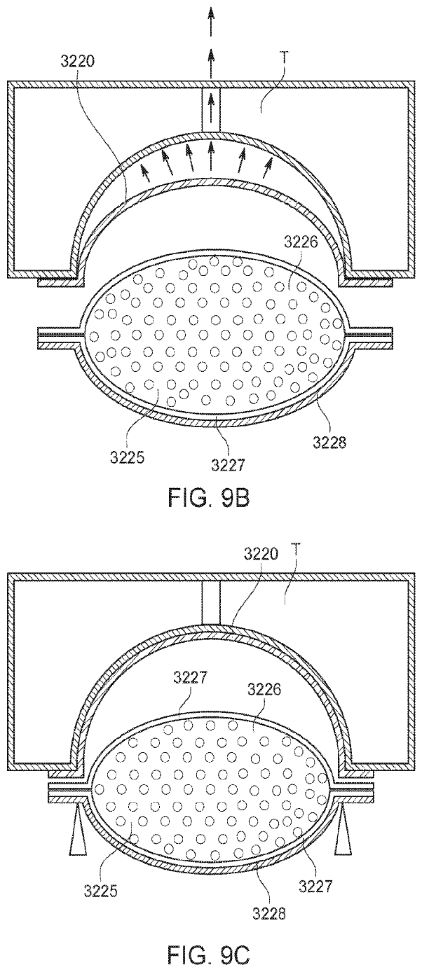

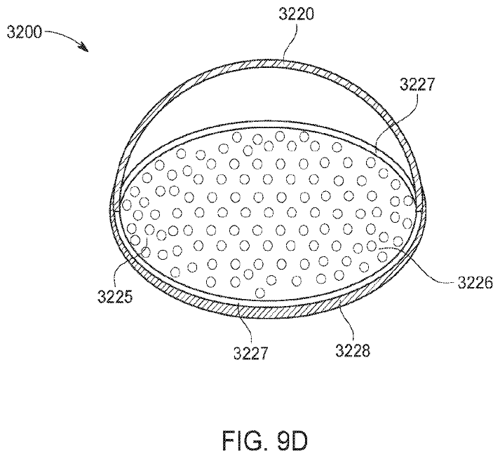

FIG. 9A-9D show schematic views of a cushion module being manufactured according to one form of the present technology.

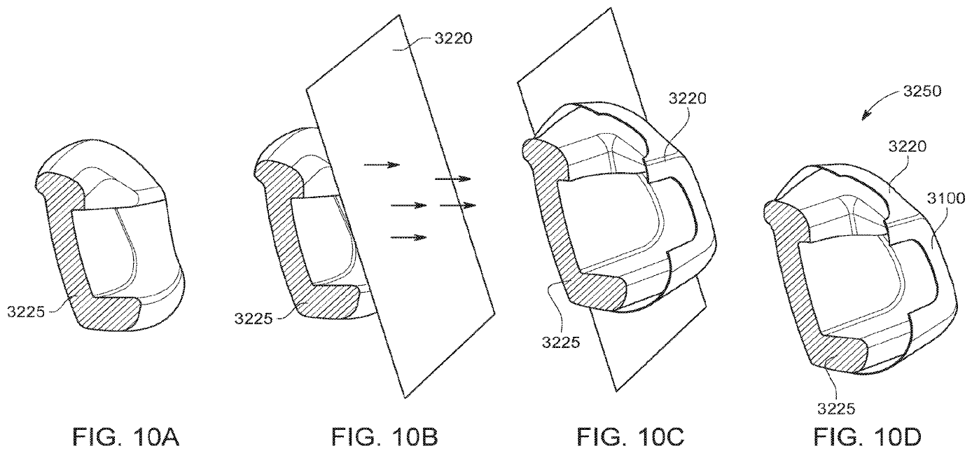

FIGS. 10A-10D show schematic views of a cushion module being manufactured according to one form of the present technology.

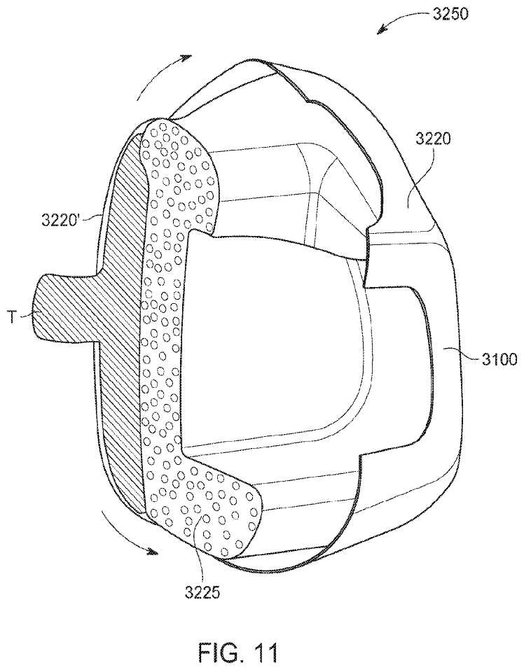

FIG. 11 shows a schematic view of a cushion module according to another form of the present technology.

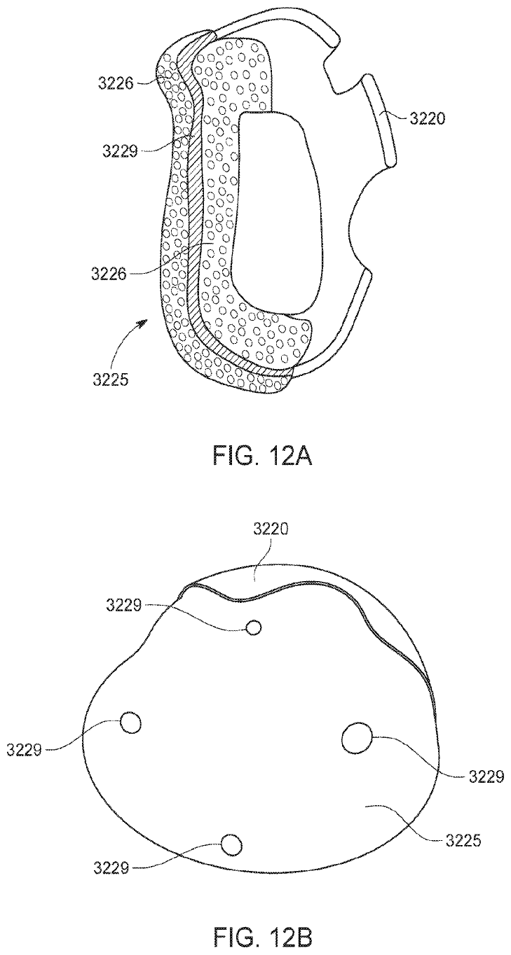

FIGS. 12A and 12B show schematic views of a cushion module according to another form of the present technology.

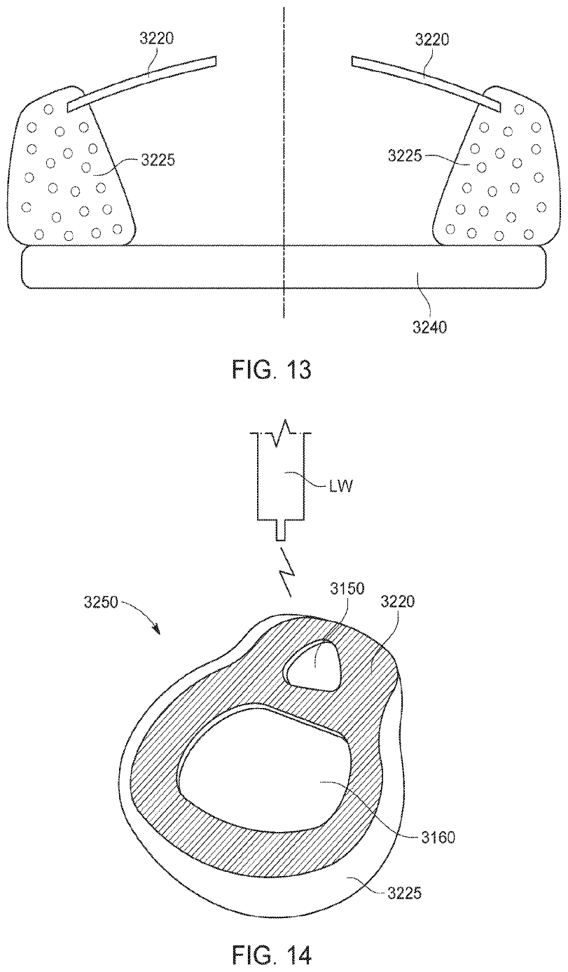

FIG. 13 shows a cross section view of a cushion module according to another form of the present technology.

FIG. 14 shows a schematic view of a cushion module according to another form of the present technology.

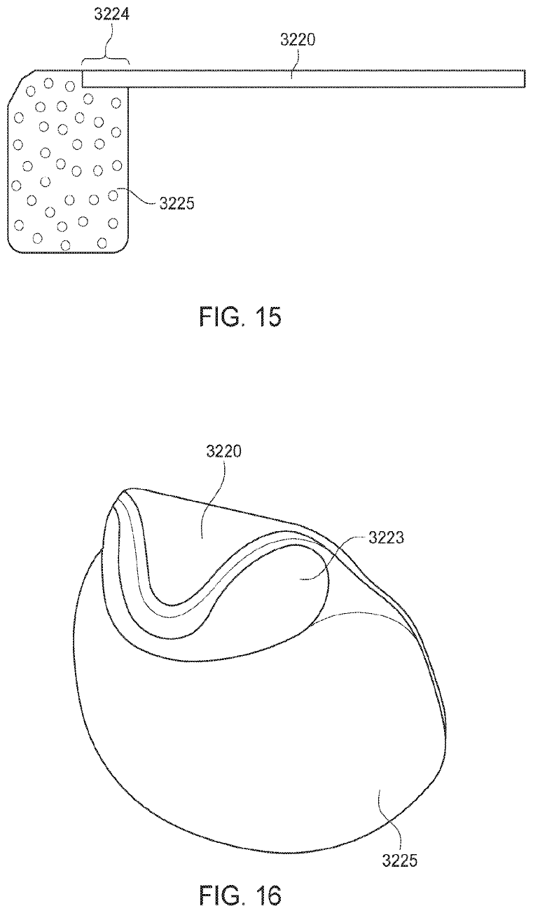

FIG. 15 shows a schematic cross section view of a cushion module according to another form of the present technology.

FIG. 16 shows a perspective view of a cushion module according to another form of the present technology.

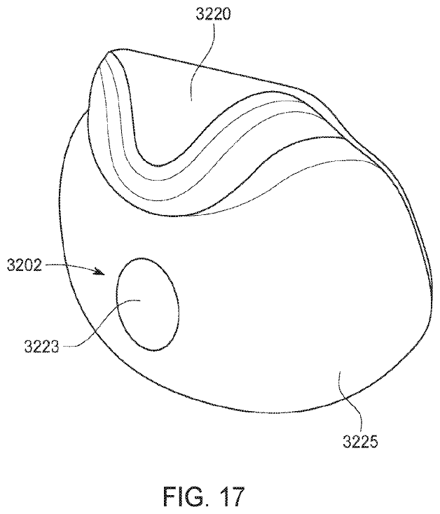

FIG. 17 shows a perspective view of a cushion module according to another form of the present technology.

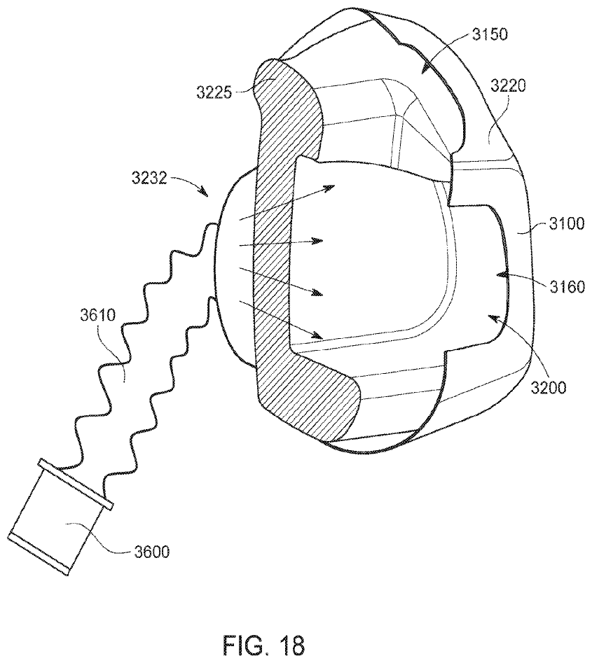

FIG. 18 shows a schematic cross section view of a cushion module according to another form of the present technology.

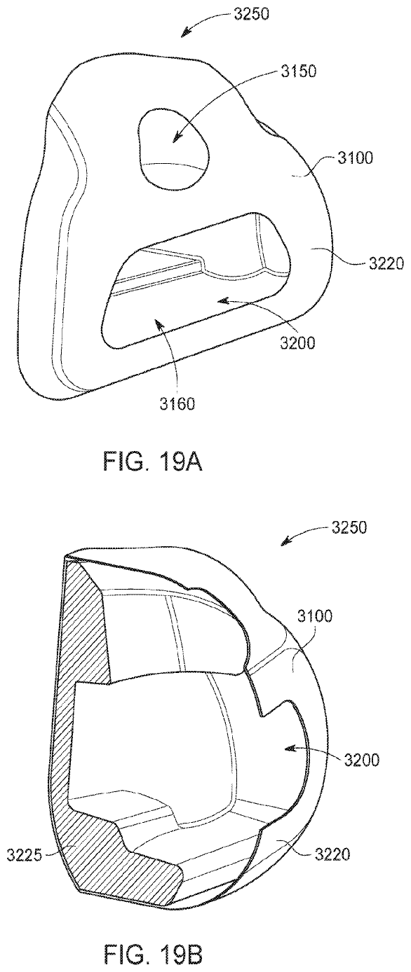

FIGS. 19A and 19B show views of a cushion module according to another form of the present technology.

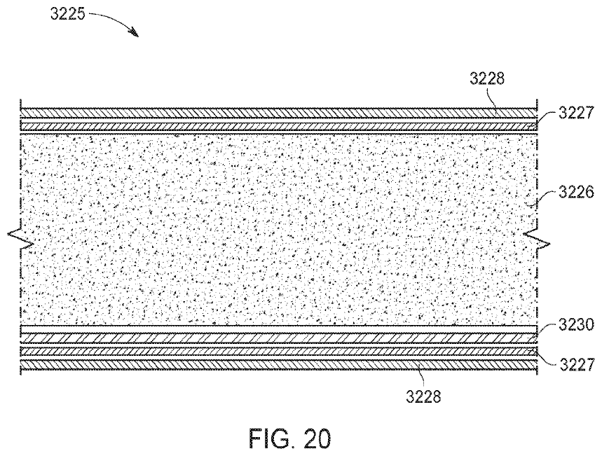

FIG. 20 shows a schematic cross section view of a chassis portion of the cushion module shown in FIGS. 19A and 19B.

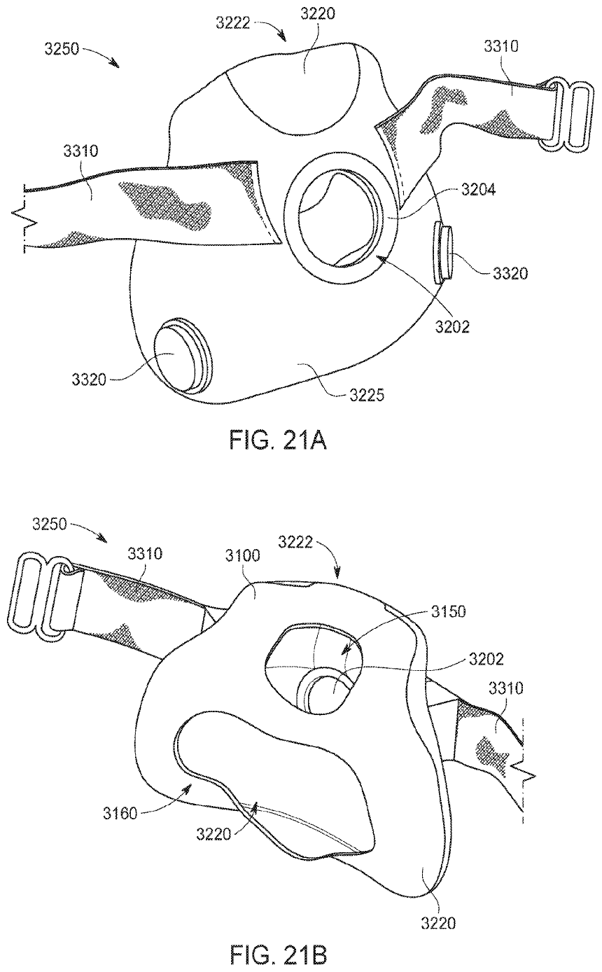

FIG. 21A shows an anterior perspective view of a cushion module according to another example of the present technology.

FIG. 21B shows a posterior perspective view of the cushion module shown in FIG. 21A.

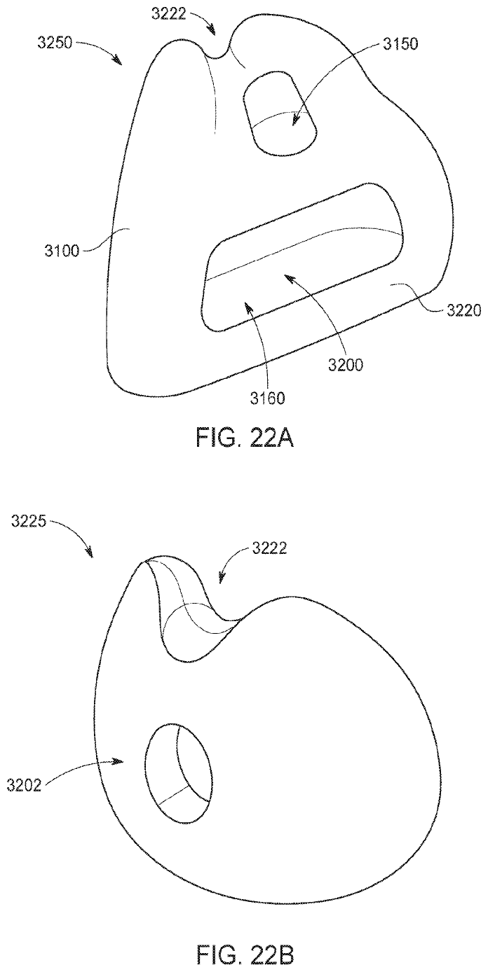

FIG. 22A shows a posterior perspective view of a cushion module according to another example of the present technology.

FIG. 22B shows an anterior perspective view of the cushion module shown in FIG. 22A.

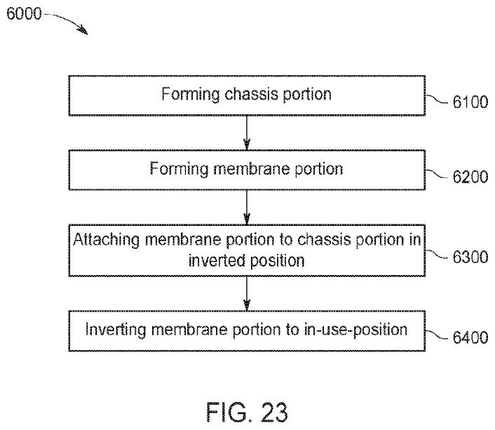

FIG. 23 shows a flow chart of a method according to another example of the present technology.

### 4 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 4.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 4.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000 or 3800.

### 4.3 Patient Interface

A non-invasive patient interface 3000 in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and, in some particular examples, a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure of at least 6 cmH₂O with respect to ambient.

The patient interface 3000 in accordance with one form of the present technology is constructed and arranged to be able to provide a supply of air at a positive pressure of at least 10 cmH₂O with respect to ambient.

The patient interface 3000 in accordance with one form of the present technology is constructed and arranged to be able to provide a supply of air at a positive pressure of at least 20 cmH₂O with respect to ambient, for example up to 30 cmH₂O or up to 40 cmH₂O.

### 4.3.1 Plenum Chamber

The plenum chamber 3200 has a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200.

The plenum chamber 3200 may comprise a plenum chamber inlet port 3202. The plenum chamber inlet port 3202 is not shown in most of FIGS. 7-20 but is shown in FIG. 21B. The plenum chamber inlet port 3202 in these examples may be a single opening provided in the chassis portion 3225 or, if the flow of air is provided by conduit headgear for example, the plenum chamber 3200 may comprise two plenum chamber inlet ports 3202 in the chassis portion 3225. In the FIG. 17 example, the chassis portion 3225 comprises a single opening forming the plenum chamber inlet port 3202. In that example the plenum chamber inlet port 3202 is filled by an insert 3223 as will be described below. In some examples, such as that shown in FIG. 18, a short tube 3610 is connected to the chassis portion 3225 and fluidly connected to the plenum chamber 3200 to convey a flow of air to the plenum chamber 3200. The short tube 3610 may comprise a connection port 3600 at a distal end thereof for connection to an air circuit 4170 to supply the flow of air from an RPT device 4000. In other examples a connector (e.g. elbow, swivel etc.) attached to the chassis portion 3225 may form a connection port 3600 for connection to an air circuit 4170. In some examples, such as that shown in FIG. 18, the plenum chamber inlet port is formed by a heat and moisture exchange portion (HMX portion) 3232 formed by the chassis portion 3225, through which the flow of air at therapeutic pressure is provided into the plenum chamber 3200, as will be described. In some examples, such as is shown in FIG. 21A, the patient interface 3000 comprises a ring 3204 defining the plenum chamber inlet port 3202. In some examples the ring 3204 may also provide a vent 3400 for the patient interface 3000, for example by defining a plurality of vent holes, such as around its circumference,

### 4.3.2 Seal-Forming Structure

In one form of the present technology, a seal-forming structure 3100 provides a target seal-forming region, and may additionally provide a cushioning function. The target seal-forming region is a region on the seal-forming structure 3100 where sealing may occur. The region where sealing actually occurs—the actual sealing surface—may change within a given treatment session, from day to day, and from patient to patient, depending on a range of factors including for example, where the patient interface 3000 was placed on the face, tension in the positioning and stabilising structure 3300 and the shape of a patient's face.

In one form the target seal-forming region is located on an outside surface of the seal-forming structure 3100.

In certain forms of the present technology, the seal-forming structure 3100 is constructed from a biocompatible material, e.g. silicone rubber. In other forms the seal forming structure 3100 comprises a foam undercushion and a textile membrane portion 3220, as described further below.

A seal-forming structure 3100 in accordance with the present technology may comprise a soft, flexible, resilient material such as silicone.

In certain forms of the present technology, a system is provided comprising more than one a seal-forming structure 3100, each being configured to correspond to a different size and/or shape range. For example the system may comprise one form of a seal-forming structure 3100 suitable for a large sized head, but not a small sized head and another suitable for a small sized head, but not a large sized head. However, examples of the technology may be suitable for a large range of heads, and so may be used by patients having a relatively large head and a relatively small head.

### 4.3.3 Cushion Module

FIGS. 7-22B show cushion modules 3250 for patient interfaces 3000 according to examples of the present technology. In some examples of the present technology, the patient interface 3000 comprises a plenum chamber 3200 pressurisable to a therapeutic pressure of at least 6 cmH₂O above ambient air pressure, the plenum chamber 3200 configured to receive a flow of air at the therapeutic pressure for breathing by a patient. The patient interface 3000 may further comprise a seal-forming structure 3100, as shown in many of FIGS. 7-22B, constructed and arranged to form a seal with a region of the patient's face surrounding an entrance to the patient's airways, the seal-forming structure 3100 having at least one hole therein such that the flow of air at the therapeutic pressure is delivered to an entrance to the patient's nares and the patient's mouth, the seal-forming structure constructed and arranged to maintain said therapeutic pressure in the plenum chamber 3200 throughout the patient's respiratory cycle in use. Many of FIGS. 7-22B show a seal-forming structure 3100 having a nasal hole 3150 constructed and arranged to deliver the flow of air to the patient's nares and an oral hole 3160 constructed and arranged to deliver the flow of air to the patient's mouth. The patient interface 3000 may further comprise a vent 3400 to allow a continuous flow of gases exhaled by the patient from an interior of the plenum chamber 3200 to ambient, said vent 3400 being sized and shaped to maintain the therapeutic pressure in the plenum chamber 3200 in use. Patient interfaces 3000 having a cushion module 3250 as shown in FIGS. 7-22B for example are configured to allow the patient to breathe from ambient through their mouth in the absence of a flow of pressurised air through the plenum chamber inlet port, for example by being provided with an anti-asphyxia valve (AAV).

In some examples of the present technology, the patient interface 3000 comprises a chassis portion 3225 and a membrane portion 3220 together at least partially forming the plenum chamber 3200. The membrane portion 3220 may be attached to the chassis portion 3225 and may form the seal-forming structure 3100. In the examples shown in FIGS. 7A-7D, FIGS. 21A-21B and 22A-22B for example, a chassis portion 3225 and a membrane portion 3220 form a cushion module 3250 for a patient interface 3000. The cushion modules 3250 in FIGS. 7A-7D and FIGS. 22A-22B may be provided with headgear connectors (not shown) for connection to a positioning and stabilising structure 3300 of the patient interface 3000. The cushion module 3250 shown in FIGS. 21A-21B includes headgear connectors in the form of superior headgear connection portions 3310 and inferior headgear connection portions 3320, which are described below.

The cushion module 3250 may also be provided with a plenum chamber inlet port 3202 and connection port 3600 (not shown) to provide for connection to an air circuit 4170. A vent 3400 (not shown) may be provided in the chassis portion 3225 or in a connector which may also form the connection port 3600.

The membrane portion 3220 may be formed at least partially from a textile material. In some examples, the membrane portion 3220 may comprise an air-impermeable layer and a textile layer. The textile layer may form a patient-facing side of the membrane portion 3220. Advantageously this may provide for a patient interface 3000 that is highly comfortable and/or at least perceived by the patient as more like an item of sleepwear than a patient interface 3000 that is formed from mostly from plastics materials, which may advantageously increase patient compliance with therapy.

### 4.3.3.1 Shape of Cushion Module

The cushion module 3250 may be constructed and arranged to engage with the patient's face such that the seal-forming structure 3100 (formed by the membrane portion 3220) forms a stable seal around an entrance to the patient's airways.

In the examples shown in many of FIGS. 7A-22B, the cushion module 3250 and the seal-forming structure 3100 thereof are constructed and arranged to seal around the entrance to the patient's nares and around the patient's mouth. The seal-forming structure 3100 is structured to engage and seal to the patient's face at the lip inferior, the cheeks, the lip superior, between the patient's nose and nasolabial sulci, to the nasal ala and to a pronasale region of the patient's nose. The patient interface 3000 having the cushion module 3250 may be identified as an ultra-compact full face (UCFF) mask.

In other forms the cushion module 3250 and seal-forming structure 3100 thereof may be constructed and arranged to seal around only the entrance to the patient's nares, for example by engaging and sealing to the patient's face at the lip superior, between the patient's nose and nasolabial sulci, and either to the nasal ala and a pronasale region of the patients nose (e.g. in the manner of a cradle cushion) or over the nasal ridge (e.g. in the manner of an over the nose nasal mask cushion).

The membrane portion 3220 may be attached to the chassis portion 3225 at or proximate a periphery of the chassis portion 3225. The chassis portion 3225 may form an anterior side of the cushion module 3250 and the membrane portion 3220 may form a posterior side of the cushion module 3250. Cushion modules 3250 having this arrangement are shown in FIGS. 7A-7D, 19A-19B and 21A-22B, for example. In some examples, the chassis portion 3225 of the cushion module 3250 may function as an undercushion and may be configured to urge the membrane portion 3220 against the patient's face in use.

With particular reference to FIGS. 21A-22B, the chassis portion 3225 may comprise a nasal recess 3222 formed in the chassis portion 3225 and located proximate a pronasale region of the patient's nose in use. The nasal recess 3222 may be recessed with respect to adjacent lateral portions of the chassis portion 3225. The nasal recess 3222 may be a region of the chassis portion 3225 shaped to receive the patient's nose, or at least the pronasale. The chassis portion 3225 may be shaped at the nasal recess 3222 to be positioned inferior to and lateral of the patient's nose or at least the pronasale thereof. The nasal recess 3222 may be shaped complementarily to the patient's nose. FIG. 22B shows the chassis portion 3225 in isolation. As illustrated, the nasal recess 3222 may comprise a saddle portion formed in the chassis portion 3225. That is, the chassis portion 3225 may comprise a saddle portion or have a saddle shape at the nasal recess 3222.

The membrane portion 3220 has a shape at the nasal recess 3222 which at least partially follows the shape of the cushion module 3250. In some examples, such as the example shown in FIG. 22A, the membrane portion 3220 is slack at the nasal recess 3222. That is, the membrane portion 3220 may not be taut. The membrane portion 3220 may be attached to the chassis portion 3225 such that the membrane portion 3220 hangs or lies in the nasal recess 3222. The cushion module 3250 in such an example is constructed and arranged to allow a pronasale region of the user's nose to lie in the nasal recess 3222 in use. Advantageously, this may reduce the pressure exerted on the patient's nose in use in comparison to a cushion module 3250 that does not have a nasal recess 3222, since the cushion module 3250 is shaped with space for the nose to be positioned in use. In some examples, the cushion module 3250 may be constructed and arranged to allow the pronasale of a patient with a long nose to protrude anteriorly of the anterior side of the chassis portion 3250. That is, not only may the cushion module 3250 have a nasal recess 3222 which is shaped to receive the nose, the nasal recess 3222 may be sufficiently deep to allow a long nose to pass over the inferior-most point of the nasal recess 3222 and protrude anteriorly of the chassis portion 3225. Advantageously, this may provide for a patient interface 3000 which can be used comfortably by patients with particularly long noses.

In other examples, the membrane portion 3220 may be taut at the nasal recess 3222. Such an example is shown in FIGS. 21A and 21B. The chassis portion 3225 in this example comprises a nasal recess 3222 in the same manner as the example shown in FIGS. 22A and 22B, but the membrane portion 3220 does not follow the saddle shape of the nasal recess 3222. Instead, the membrane portion 3220 spans across the nasal recess 3222 from one lateral side of the nasal recess 3222 to the other lateral side of the nasal recess 3222 leaving space between the membrane portion 3220 and the chassis portion 3225 at the nasal recess 3222. While it is described above that a membrane portion 3220 which is slack and allows the user's nose to pass through the nasal recess 3222, the example shown in FIGS. 21A-21B may provide a solution for patients with short to moderate length noses. The nasal recess 3222 formed in the chassis portion 3225 may still advantageously prevent excessive pressure from being applied to the patient's nose in use, at least for patients without a particularly long nose. The membrane portion 3220 may be constructed and arranged to be deformed into or towards the shape of the nasal recess 3222 by the patient's nose in use. In other examples, the chassis portion 3225 may not comprise a nasal recess 3222. Such an example may advantageously be suited to patient's having short and wide noses.

The membrane portion 3220 may a first hole through which air can flow to both the patient's nares, in use, and may comprise a second hole through which air can flow to the patient's mouth, in use. As shown in FIGS. 7B, 21B and 22A, among others, the membrane portion 3220 comprises a nasal hole 3150 for delivering a flow of air from the plenum chamber 3200 to the patient's nares, and an oral hole 3160 for delivering the flow of air from the plenum chamber 3200 to the patient's mouth. In other examples the membrane portion 3220 may comprise a single hole delivering the flow to the patient's nose and mouth. In further examples the membrane portion 32220 may comprise two nasal holes 3150, each delivering the flow of air to a respective naris, and an oral hole 3160.

### 4.3.3.2 Manufacturing of Cushion Module

Described below are various examples of how the chassis portion 3225 and the membrane portion 3220 may each be manufactured. It is to be understood that, unless context requires otherwise, any chassis portion 3225 described herein may be combined with any membrane portion 3220 described herein. It is to be understood that each example of a chassis portion 3225 described herein may be combined with a membrane portion 3220 formed at least partially from a textile material or may be combined with a membrane portion 3220 formed from an elastomeric material such as silicone or a TPE, unless requires otherwise. As generalisable examples, the chassis portion 3225 may be formed by thermoforming, may be formed by injection moulding, may be formed by cutting of foam, for example compression cutting, or may be formed by another suitable process. The foam material may be a polyurethane form, for example.

In examples in which the membrane portion 3220 is formed from an elastomeric material, the membrane portion 3220 may, for example, be moulded and attached to the chassis portion 3225 or may be overmoulded to the chassis portion 3225. In examples in which the membrane portion 3220 is formed at least partially from a textile material, the membrane portion 3220 may be formed by knitting, weaving or the like and/or may be formed by lamination of a textile layer (e.g. formed by knitting, weaving or the like) and an air-impermeable layer (e.g. a TPU film or a film formed from another suitable material for bonding with and providing air-impermeability to the textile layer). Some non-limiting specific examples are provided below.

### 4.3.3.2.1 Injection Moulded Chassis Portion

In some forms of the present technology, the chassis portion 3225 is formed by injection moulding. In some examples, the chassis portion 3225 may be formed from a foam material 3226 and a covering 3227 provided to at least one portion of the foam material 3326. The chassis portion 3225 may be formed by injection moulding of the foam material 3226 against the covering 3226. FIG. 8 shows a schematic of a chassis portion 3225 in one example of this form of the present technology.

As shown in FIG. 8, the covering 3227 may be provided on two opposite sides of the chassis portion 3225. For example, the covering 3227 may comprise an anterior layer on an anterior side of the chassis portion 3225 and a posterior layer on a posterior side of the chassis portion 3225. As shown in FIG. 8, the foam material 3226 is covered by two layers of a covering 3227 on opposite sides. In this particular example, the chassis portion 3225 is formed by injection moulding of the foam material 3226 between the anterior layer and the posterior layer of the covering 3227. In some examples the anterior layer and the posterior layer are integrally formed with each other (e.g. the foam material 3226 may be injected into one of more holes in an integrally formed covering 3227 to result in foam material 3226 substantially encapsulated by the covering 3227). In other examples the anterior and posterior layers of the covering 3227 may be attached to each other before or after injection moulding. In the FIG. 8 example two separately formed layers forming the covering 3227 are held in place and the foam material 3226 is injection moulded between them. The layers may be joined in a subsequent step after injection moulding, for example by RF, ultrasonic or laser welding or other heat bonding process. One or both of the anterior and posterior layers forming the covering 3227 may comprise a textile layer and/or an air-impermeable layer. In other examples only one side of the foam material 3226 may be covered with a covering 3227. The foam material 3226 may comprise a polyurethane foam, for example.

The membrane portion 3220 and the chassis portion 3225 may be formed separately. The membrane portion 3220 may be attached to the chassis portion 3225 after the chassis portion 3225 at or proximate a periphery of the chassis portion 3225. As shown in FIG. 9A-9D or 10A-10D for example, the membrane portion 3220 may be attached to a periphery of the chassis portion 3225 after the chassis portion 3225 has been formed. The membrane portion 3220 may be attached to a periphery of the chassis portion 3225 while the membrane portion 3220 is under tension. This may advantageously result in the membrane portion 3220 being taut after connection to the chassis portion 3225.

In some particular examples, the membrane portion 3220 may be attached to the chassis portion 3225 in an inverted position in which the membrane portion 3220 is positioned on an anterior side of the chassis portion 3225. The membrane portion 3220 may then be inverted to an in-use position in which the membrane portion 3220 is positioned on a posterior side of the chassis portion 3225. This may advantageously provide for a soft feeling and aesthetically pleasing connection between the membrane portion 3220 and the chassis portion 3225 due to the seam/connection between the membrane portion 3220 and the chassis portion 3225 being hidden by the inverted membrane portion 3220. It may also allow for a patient contacting layer of the membrane portion 3220 to be the layer that forms a bond with the chassis portion 3225, which may be desirable where a backing layer, such as an air-impermeable layer, is formed from a material that does not bond well or easily to the chassis portion 3225. In some examples the membrane portion 3220 may comprise a textile patient-contacting layer, which may bond well to the chassis portion 3225, and a silicone backing layer, which may not bond as well or as easily to the chassis portion 3225 as the textile layer does. Bonding the textile layer to the chassis portion 3225 and then inverting (flipping inside out) the membrane portion 3220 allows the textile layer to form both the patient-contacting surface and an attachment surface of the membrane portion 3220.

In some examples, the membrane portion 3220 may be attached to the chassis portion 3220 by one of RF welding, ultrasonic welding and laser welding, for example. As shown in FIG. 14, the membrane portion 3220 may be placed onto a chassis portion 3225 and the periphery of the membrane portion 3220 may be laser welded to the chassis portion 3220 by a laser welding tool LW, for example proximate the periphery of the chassis portion as shown in FIG. 14.

In other examples, the membrane portion 3220 may be adhered to the chassis portion 3225. The chassis portion 3225 may comprise a flat portion 3224, as shown in FIG. 15 for example, having a shape and size corresponding to the shape and size of a periphery of the membrane portion 3220, the periphery of the membrane portion 3220 being adhered to the flat portion 3224. The membrane portion 3220 in this particular example is adhered to the chassis portion with a thermoset material. The thermoset material may be silicone for example. In other examples any other suitable glue or component which can function as an adhesive could be used.

### 4.3.3.2.2 Chassis Portion Overmoulded to Membrane Portion

In some forms of the present technology, the chassis portion 3225 is formed from a foam material 3226 and is formed by injection moulding of the foam material 3226. The foam material 3226 may be overmoulded to the membrane portion 3220. FIG. 13 shows one such example. The membrane portion 3220 may be attached to a posterior side of the chassis portion 3225 after the overmoulding step, for example. As shown in FIG. 13, the foam material 3226 may be overmoulded to the membrane portion 3220 such that the membrane portion 3220 is attached to an inwardly facing surface on a posterior side of the chassis portion 3225. In the FIG. 13 example the chassis portion is attached to a frame 3240 which may reinforce the chassis portion 3225, provide headgear connections and/or provide a connection to an air circuit 4170, by way of example only.

In another example, the foam material 3226 may be overmoulded to the membrane portion 3220 such that the membrane portion 3220 is attached to the chassis portion 3220 at or proximate a periphery of the chassis portion 3225. The membrane portion 3220 may be inverted after attachment to the anterior surface of the chassis portion 3225 to provide the seal-forming structure 3100 on a posterior side of the chassis portion 3225 (advantages of which are described above). FIG. 11 shows an example of this configuration. The membrane portion 3220 may be held in the position indicated by 3220′ by tool T. The chassis portion 3225 may be formed by injection moulding on the opposite side of tool T and in the process may be overmoulded to the peripheral edges of the membrane portion 3220. The cushion module 3250 may then be removed from the tool T and the membrane portion 3220 may be inverted from the position indicated by 3220′ on the anterior side of the chassis portion 3225 to the position indicated by 3220 on the posterior side of the chassis portion 3225 to form a seal-forming structure 3100 on the posterior side of the chassis portion 3225. The membrane portion 3220 may be held under tension in the position indicated by 3220′ such that it takes a predetermined desired shape after the inversion step. The foam material 3226 may be a polyurethane foam in this example.

In further examples, the chassis portion 3225 may be formed in isolation by injection moulding or thermoforming of the foam material 3226, for example as described above or elsewhere herein. The membrane portion 3220 may be attached to the chassis portion 3220 at or proximate a periphery of the chassis portion 3225, for example by RF, ultrasonic or laser welding, or gluing. The membrane portion 3220 may be attached while positioned on the posterior side or may be attached while on the anterior side of the chassis portion 3225, after which it may be inverted to form the seal-forming structure 3100 on the posterior side. In some examples the membrane portion 3220 comprises an air impermeable layer, which may for example be formed from TPU which may be bonded sufficiently easily to the chassis portion 3225 and may not need inversion.

### 4.3.3.2.3 Membrane Portion and Chassis Portion Formed Separately

The chassis portion 3225 and membrane portion 3220 in some examples are formed separately. The chassis portion 3225 may be formed at least partially from a foam material 3226 and the membrane portion 3220 may be formed at least partially from a textile material 3220. In such an example, the membrane portion 3220 may be attached to the chassis portion 3225 in any suitable manner. In some examples the membrane portion 3220 may be attached the chassis portion 3225 in an inverted position in which the membrane portion 3220 is positioned on an anterior side of the chassis portion 3225. The membrane portion 3220 may then be inverted to an in-use position in which the membrane portion 3220 is positioned on a posterior side of the chassis portion 3225.

Described differently, the membrane portion 3220 may have a patient-facing surface which faces and engages the patient's face in use and, in the inverted position during attachment of the membrane portion 3220 to the chassis portion 3225, at least a majority of the patient-facing surface of the membrane portion 3220 may be positioned anteriorly of the chassis portion 3225 and may face anteriorly. After inversion from the inverted position to an in-use position, the patient-facing surface of the membrane portion 3220 may face posteriorly and may be positioned posteriorly of the chassis portion 3225.

In some examples, the chassis portion 3225 is thermoformed, e.g. formed by thermoforming. FIGS. 19A-19B, 20, 21A-21B and 22A-22B show examples of thermoformed chassis portions 3225. The chassis portion 3225 may comprise an air-impermeable layer provided to at least one of an anterior side and a posterior side of the foam material 3226. The cross-sectional arrangement/layering of materials shown in FIG. 20 is to be understood to be applicable to any example described herein, unless the context requires otherwise. As shown in FIG. 20, the cushion module 3250 in this example comprises a covering 3227 on both anterior and posterior sides, which may be an air-impermeable layer. The air-impermeable layer may comprise a TPU film, which could be 0.05 mm in thickness for example. In other examples the air-impermeable layer may be formed from another suitable material, such as a silicone film or thermoplastic elastomer (TPE) film. The chassis portion 3225 may comprise a textile layer 3228 on at least one of the anterior side and the posterior side of the chassis portion 3220. In the FIGS. 19A-19B and FIG. 20 example the chassis portion 3220 comprises a textile layer 3228 on each of the posterior and anterior sides. This may be a 0.25 mm layer and may be formed from a 4-way stretch fabric, for example. Any suitable material, knitting pattern and thickness may be selected for the textile layer 3228. The chassis portion 3225 may further comprise a stiffening layer 3230 on an anterior side of the chassis portion, as shown in FIG. 20. The stiffening layer 3230 may for example be formed from a material stiffer than the foam material forming the chassis portion 3225. In other examples a stiffening layer 3230 may be provided on the posterior side or on both anterior and posterior sides of the chassis portion 3225. The stiffening layer 3230 may reinforce the chassis portion 3225 and may be a suitable thermoplastic material such as polycarbonate, TPU/TPE or PET, for example. In some examples the thickness of the stiffening layer is less than 0.5 mm. In some examples the stiffening layer may be 0.3 mm in thickness. The stiffening layer 3230 may be a film.

In some examples, the stiffening layer 3230 covers substantially the whole anterior side of the chassis portion 3225. In other examples the stiffening layer 3230 may be provided to one or more regions of the chassis portion 3225. In one example, the stiffening layer 3230 is provided to the centre of the anterior side of the chassis portion 3225 and comprises projecting portions extending to peripheral portions of the chassis portion 3225 proximate headgear connections. This shape may be identified as a “spider shape”. The centre of such shape may be proximate a plenum chamber inlet port 3202 and may have four “legs” that extend to the superior headgear connection portions 3310 and inferior headgear connection portions 3320, respectively.

The chassis portion 3225 may alternatively be formed in isolation by injection moulding of the foam material 3226, as described above.

FIGS. 9A-9D show another example of construction of a cushion module 3250 comprising a chassis portion 3225 and a membrane portion 3220. The chassis portion 3225 and membrane portion 3220 may be formed separately of each other and then attached together in this example. The chassis portion 3225 may be formed by injection moulding or thermoforming, for example. In the particular example shown by FIG. 9A-9D, the chassis portion 3225 is formed by injection moulding in the manner described with reference to FIG. 8. The chassis portion 3225 in this example also comprises a textile layer 3228 on a non-patient facing side (e.g. an anterior side). As shown in FIGS. 9A-9D, once the chassis portion 3225 is formed (FIG. 9A), the membrane portion 3220 is held by a tool T and brought into contact with the chassis portion 3225 (FIGS. 9B and 9C). Finally, in this particular example there is excess material from each of the layers formed by the covering 3227, textile layer 3228 and membrane portion 3220. As shown in FIGS. 9C and 9D the excess material is removed, for example by an RF, ultrasonic or heat cutting process. In some examples, this cutting process may also bond the layers together, for example bonding the membrane portion 3220 layer to the chassis portion 3225 and in some examples bonding the layers of the chassis portion 3225 together. In other examples, the layers may be attached (e.g. by welding, adhesive or another bonding operation) and then any excess may be cut. The membrane portion 3220 when held by the tool T may be held in shape by a vacuum and also may be heated. FIGS. 10A-10D also illustrate the process of the membrane portion 3220 being applied to the chassis portion 3225 from sheet form as shown in FIG. 10B to the form of the seal-forming structure 3100 as shown in FIG. 10D.

As described above with reference FIGS. 21A-22B, in some examples the chassis portion 3225 comprises a nasal recess 3222 and the membrane portion 3220 may be slack at the nasal recess 3222. The process described above with reference to FIGS. 9A-9D may be applied to provide the membrane portion 3220 with a slack portion (e.g. looseness) at the nasal recess 3222, to accommodate long noses within the nasal recess 3222 in use. In some such examples, the membrane portion 3220 is attached to the chassis portion 3225 (e.g. as described above with reference to FIGS. 9A-9D) while held in a predetermined shape by vacuum during attachment to the chassis portion 3225. The membrane portion 3220 may be attached to the chassis portion 3225 while under tension. For example, the membrane portion 3220 is stretched in a nasal region of the membrane portion 3220 while held in the predetermined shape during attachment such that the nasal region of the membrane portion 3220 is slack when in the in-use position. That is, the vacuum may both hold the membrane portion 3220 during attachment to the chassis portion 3225 and also stretch the membrane portion 3220 so that after inversion, the membrane portion 3220 has some looseness in the nasal region so that the membrane portion 3220 will lie within and follow the shape of a nasal recess 3222 in the chassis portion 3225.

Accordingly, one aspect of the present technology is a method 6000 of manufacturing a cushion module 3250 of a patient interface 3000. FIG. 23 shows a flow chart summarising the method 6000. Each step may have a plurality of sub-steps. The method 6000 may comprise forming the chassis portion 3225 of the cushion module 3250 and the membrane portion 3220 of the cushion module 3250 separately. Step 6100 is forming the chassis portion 3225 and step 6200 is forming the membrane portion 3220. These first two steps may be performed in any order or simultaneously. The chassis portion 3250 and the membrane portion 3220 may be constructed and arranged to together at least partially form a plenum chamber 3200 of the patient interface 3000, the plenum chamber 3200 being pressurisable to a therapeutic pressure of at least 6 cmH₂O above ambient air pressure and being configured to receive a flow of air at the therapeutic pressure for breathing by a patient.

The method 6000 may comprise, at step 6300, attaching the membrane portion 3220 to the chassis portion 3225 in an inverted position. In the inverted position, the membrane portion 3220 is positioned on an anterior side of the chassis portion 3225. The method 6000 may further comprise, at step 6400 inverting the membrane portion 3220 to an in-use position in which the membrane portion 6220 is positioned on a posterior side of the chassis portion 3225. In the in-use position, the membrane portion 3220 forms a seal-forming structure 3100 of the patient interface 3000, as described elsewhere herein.

The step 6100 of forming the chassis portion 3225 may comprise forming the chassis portion 3225 at least partially from a foam material. In some examples, the step 6100 comprises thermoforming the chassis portion 3225. In other examples, the step 6100 comprises injection moulding the chassis portion 3225.

The step 6200 of forming the membrane portion 3220 may comprise forming the membrane portion 3220 at least partially from a textile material. For example, the step 6200 may comprise laminating a textile layer and an air impermeable layer to form the membrane portion 3220. In other examples, the step 6200 may comprise forming the plenum chamber 3200 from an elastomeric material, for example by moulding.

The step 6300 of attaching the membrane portion 3220 to the chassis portion 3225 may comprise attaching the membrane portion 6220 to the chassis portion 3225 at or proximate a periphery of the chassis portion 3225. In some examples, as has been described above, the step of attaching the membrane portion 3220 to the chassis portion 3225 comprises holding the membrane portion 3220 in a predetermined shape by vacuum during attachment to the chassis portion 3225. This step 6300 may comprise tensioning at least a portion of the membrane portion 3220 during attachment. For example, step 6300 may comprise stretching the membrane portion 3220 in a nasal region of the membrane portion 3220 while held in the predetermined shape during attachment such that the nasal region of the membrane portion 3220 is slack when in the in-use position. Advantageously, this provides for the configuration shown in FIG. 22A in which the chassis portion 3225 comprises a nasal recess 3222 and the membrane portion 3220 has a slack portion which loosely lies within the nasal recess 3222 to accommodate the patient's nose.

In some examples, the step 6300 of attaching the membrane portion 3220 to the chassis portion 3225 may comprise welding the membrane portion 3220 to the chassis portion 3225. For example, step 6300 may comprise one of RF welding, ultrasonic welding or laser welding the membrane portion 3220 to the chassis portion 3225.

The final step 6400 of the method 6000 comprises inverting the membrane portion 3220 to the in-use position, as described elsewhere herein. This step 6400 positions the membrane portion 3220 on a posterior side of the chassis portion 3225 to form a seal-forming structure 3100. This step 6400 may be performed in any suitable manner, for example by hand or by machine, e.g. by robot.

### 4.3.3.2.4 Chassis Portion Formed from Two Portions

FIGS. 12A and 12B show a cushion module 3250 according to another example of the present technology, in which the chassis portion 3225 comprises a first portion 3229 and a second portion comprising a foam material 3226 attached to the first portion 3229. The membrane portion 3220 in this example is attached to the first portion 3229.

The second portion may be formed by injection moulding of the foam material 3226. The foam material 3226 may be overmoulded to the first portion 3229. The foam material 3226 may be overmoulded to both an anterior side and a posterior side of the first portion 3229 as shown in FIG. 12A. The foam material 3226 may at least partially encapsulate the first portion 3229 and in some examples may substantially completely encapsulate it. The first portion 3229 may be constructed and arranged to stiffen the chassis portion 3225. The first portion 3229 may be semi-rigid in some examples. The first portion 3229 may be formed from TPE or TPU or another suitable material. In some examples it may be formed from silicone. In further examples it may be formed from an even stiffer material such as Hytrel or polycarbonate, by way of example only. The membrane portion 3220 may be at least partially formed from a textile material and may comprise an air impermeable layer, as described elsewhere herein.

### 4.3.3.3 Inserts

FIGS. 16 and 17 show cushion modules 3250 according to further examples of the present technology. In these examples the cushion module 3250 comprises an insert 3223 configured to attach to or fit within a portion of the chassis portion 3225. In some examples, the insert 3223 is formed from a foam material. By way of example only, the foam material may be a silicone foam or a TPU foam.

As shown in FIG. 16, the insert 3223 may be insertable into an anterior side of the chassis portion 3225. The insert 3223 may be configured to reinforce the chassis portion 3225 and may be more rigid than the material forming other parts of the chassis portion 3225. The insert 3223 may be configured to function as an undercushion. The insert 3223 may be configured to support the membrane portion 3220 to seal between the patient's nasal ala and nasolabial sulci, for example.

In the example shown in FIG. 17, the insert is insertable into an interior of the plenum chamber 3200. In this example the insert 3223 may be air-permeable and may be configured to block the plenum chamber inlet port to diffuse the flow of air supplied to the plenum chamber 3200. The insert may function as a windbreak, and may be formed from a sufficiently air permeable foam, for example.

### 4.3.3.4 Heat and Moisture Exchange (HMX)

In the example shown in FIG. 18, the chassis portion is at least partially formed from a material forming a heat and moisture exchange portion (HMX portion) 3232 functioning as a heat and moisture exchanger (HMX). The flow of air at the therapeutic pressure is provided through the HMX portion 3232 to enter the plenum chamber 3200, and the vent 3400 is positioned such that the flow of gases exhaled by the patient pass through the HMX portion 3232 prior to flowing through the vent 3400. The chassis portion 3225 and HMX portion 3232 may be integrally formed from foam, in some examples. The chassis portion 3225 may comprise compression cut foam, as one example only. In other examples the chassis portion 3225 may be formed by injection moulding of a foam material. The foam material forming at least the HMX portion 3232 may be open cell foam.

### 4.3.4 Positioning and Stabilising Structure

The seal-forming structure 3100 of the patient interface 3000 of the present technology may be held in sealing position in use by a positioning and stabilising structure 3300.

In one form the positioning and stabilising structure 3300 provides a retention force at least sufficient to overcome the effect of the positive pressure in the plenum chamber 3200 to lift off the face.

In one form the positioning and stabilising structure 3300 provides a retention force to overcome the effect of the gravitational force on the patient interface 3000.

In one form the positioning and stabilising structure 3300 provides a retention force as a safety margin to overcome the potential effect of disrupting forces on the patient interface 3000, such as from tube drag, or accidental interference with the patient interface.

In one form of the present technology, a positioning and stabilising structure 3300 is provided that is configured in a manner consistent with being worn by a patient while sleeping. In one example the positioning and stabilising structure 3300 has a low profile, or cross-sectional thickness, to reduce the perceived or actual bulk of the apparatus. In one example, the positioning and stabilising structure 3300 comprises at least one strap having a rectangular cross-section. In one example the positioning and stabilising structure 3300 comprises at least one flat strap.

In one form of the present technology, a positioning and stabilising structure 3300 is provided that is configured so as not to be too large and bulky to prevent the patient from lying in a supine sleeping position with a back region of the patient's head on a pillow.

In one form of the present technology, a positioning and stabilising structure 3300 is provided that is configured so as not to be too large and bulky to prevent the patient from lying in a side sleeping position with a side region of the patient's head on a pillow.

In one form of the present technology, a positioning and stabilising structure 3300 is provided with a decoupling portion located between an anterior portion of the positioning and stabilising structure 3300, and a posterior portion of the positioning and stabilising structure 3300. The decoupling portion does not resist compression and may be, e.g. a flexible or floppy strap. The decoupling portion is constructed and arranged so that when the patient lies with their head on a pillow, the presence of the decoupling portion prevents a force on the posterior portion from being transmitted along the positioning and stabilising structure 3300 and disrupting the seal.

In one form of the present technology, a positioning and stabilising structure 3300 comprises a strap constructed from a laminate of a fabric patient-contacting layer, a foam inner layer and a fabric outer layer. In one form, the foam is porous to allow moisture, (e.g., sweat), to pass through the strap. In one form, the fabric outer layer comprises loop material to engage with a hook material portion.

In certain forms of the present technology, a positioning and stabilising structure 3300 comprises a strap that is extensible, e.g. resiliently extensible. For example the strap may be configured in use to be in tension, and to direct a force to draw a seal-forming structure into sealing contact with a portion of a patient's face. In an example the strap may be configured as a tie.

In one form of the present technology, the positioning and stabilising structure comprises a first tie, the first tie being constructed and arranged so that in use at least a portion of an inferior edge thereof passes superior to an otobasion superior of the patient's head and overlays a portion of a parietal bone without overlaying the occipital bone.

In one form of the present technology suitable for a nasal-only mask or for a full-face mask, the positioning and stabilising structure includes a second tie, the second tie being constructed and arranged so that in use at least a portion of a superior edge thereof passes inferior to an otobasion inferior of the patient's head and overlays or lies inferior to the occipital bone of the patient's head.

In one form of the present technology suitable for a nasal-only mask or for a full-face mask, the positioning and stabilising structure includes a third tie that is constructed and arranged to interconnect the first tie and the second tie to reduce a tendency of the first tie and the second tie to move apart from one another.

In certain forms of the present technology, a positioning and stabilising structure 3300 comprises a strap that is bendable and e.g. non-rigid. An advantage of this aspect is that the strap is more comfortable for a patient to lie upon while the patient is sleeping.

In certain forms of the present technology, a positioning and stabilising structure 3300 comprises a strap constructed to be breathable to allow moisture vapour to be transmitted through the strap,

In certain forms of the present technology, a system is provided comprising more than one positioning and stabilizing structure 3300, each being configured to provide a retaining force to correspond to a different size and/or shape range. For example the system may comprise one form of positioning and stabilizing structure 3300 suitable for a large sized head, but not a small sized head, and another. suitable for a small sized head, but not a large sized head.

### 4.3.5 Connectors for Positioning and Stabilising Structure

Connectors or connection portions for connecting the positioning and stabilising structure 3300 to the patent interface 3000 may be provided to the chassis portion 3225 or to a frame 3240 to which the chassis portion 3225 is connected. The cushion module 3250 or frame 3240 may be provided with a pair of superior headgear connection portions 3310 and a pair of inferior headgear connection portions 3320, for example.

In particular, the cushion module 3250 may comprise headgear connection portions connected to the chassis portion 3225, the headgear connection portions constructed and arranged to connect to a positioning and stabilising structure 3300 of the patient interface 3000. As shown in FIGS. 21A-21B, the headgear connection portions comprise a pair of superior headgear connection portions 3310 constructed and arranged to connect to superior strap portions of the positioning and stabilising structure 3300 of the patient interface 3000. Also as shown in FIGS. 21A-21B, the headgear connection portions comprise a pair of inferior headgear connection portions 3320 constructed and arranged to connect to inferior strap portions of the positioning and stabilising structure 3300 of the patient interface 3000.

Each of the superior headgear connection portions 3310 may comprise a curved arm. In some examples the superior headgear connection portions 3310 each comprise a rigidised arm, each arm provided with a strap engagement means (e.g. a loop or a slot) for engaging a headgear strap at or proximate the end thereof. In some examples the inferior headgear connection portions 3320 comprise magnetic connectors, which may engage complementary connectors attached to headgear straps.

The rigidised arms may extend laterally from the cushion module 3250 or frame 3240 and then curve to extend posteriorly in use. The arms may also curve towards a superior direction, in use.

In some examples the chassis portion 3225 may comprise strap portions connected to and extending laterally from the anterior side of the chassis portion 3225. The strap portions of the chassis portion 3225 may form the superior headgear connection portions 3310 and may each be structured to connect to a respective superior strap portion of the positioning and stabilising structure 3300. The superior headgear connection portions 3310 shown in FIGS. 21A-21B take this form. The inferior headgear connection portions 3320 shown in this example are in the form of connection points constructed to magnetically connect to inferior strap portions of a positioning and stabilising structure 3300. The inferior headgear connection portions 3320 may comprise magnets or may comprise ferromagnetic material able to form a magnetic connection with magnets attached to inferior strap portions of the positioning and stabilising structure 3300. This magnetic connection of the lower headgear straps to the cushion module 3250 may allow for “set-and-forget” adjustment of the headgear straps of the positioning and stabilising structure 3300, whereby the patient or their clinician/technician adjusts the lengths of the headgear straps during fitting, after which the patient is able to easily disconnect and reconnect the lower straps from the cushion module 3250 during donning and doffing without the need to adjust strap lengths. In other examples the inferior headgear connection portions 3320 may comprise hooks, buckles, strap portions, or any other suitable connector for connection with lower headgear straps.

### 4.3.6 Vent

In one form, the patient interface 3000 includes a vent 3400 constructed and arranged to allow for the washout of exhaled gases, e.g. carbon dioxide.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO₂ by the patient while maintaining the therapeutic pressure in the plenum chamber in use.

One form of vent 3400 in accordance with the present technology comprises a plurality of holes, for example, about 20 to about 80 holes, or about 40 to about 60 holes, or about 45 to about 55 holes.

The vent 3400 may be located in the plenum chamber 3200. Alternatively, the vent 3400 is located in a decoupling structure, e.g., a swivel.

### 4.3.7 Decoupling Structure(s)

In one form the patient interface 3000 includes at least one decoupling structure, for example, a swivel or a ball and socket.

### 4.3.8 Connection Port

Connection port 3600 allows for connection to the air circuit 4170.

### 4.3.9 Forehead Support

In one form, the patient interface 3000 includes a forehead support 3700.

### 4.3.10 Anti-Asphyxia Valve

In one form, the patient interface 3000 includes an anti-asphyxia valve.

### 4.3.11 Ports

In one form of the present technology, a patient interface 3000 includes one or more ports that allow access to the volume within the plenum chamber 3200. In one form this allows a clinician to supply supplementary oxygen. In one form, this allows for the direct measurement of a property of gases within the plenum chamber 3200, such as the pressure.

### 4.4 RPT Device

An RPT device 4000 in accordance with one aspect of the present technology comprises mechanical, pneumatic, and/or electrical components and is configured to execute one or more algorithms, such as any of the methods, in whole or in part, described herein. The RPT device 4000 may be configured to generate a flow of air for delivery to a patient's airways, such as to treat one or more of the respiratory conditions described elsewhere in the present document.

In one form, the RPT device 4000 is constructed and arranged to be capable of delivering a flow of air in a range of −20 L/min to +150 L/min while maintaining a positive pressure of at least 6 cmH₂O, or at least 10cmH₂O, or at least 20 cmH₂O.

The RPT device may have an external housing 4010, formed in two parts, an upper portion 4012 and a lower portion 4014. Furthermore, the external housing 4010 may include one or more panel(s) 4015. The RPT device 4000 comprises a chassis 4016 that supports one or more internal components of the RPT device 4000. The RPT device 4000 may include a handle 4018.

The pneumatic path of the RPT device 4000 may comprise one or more air path items, e.g., one or more air filters 4110 such as an inlet air filter 4112 and/or outlet air filter 4114, an inlet muffler 4122, a pressure generator 4140 capable of supplying air at positive pressure (e.g., a blower 4142 comprising a motor 4144), one or more mufflers 4120 such as an outlet muffler 4124 and one or more transducers 4270, such as pressure sensors and flow rate sensors.

One or more of the air path items may be located within a removable unitary structure which will be referred to as a pneumatic block 4020. The pneumatic block 4020 may be located within the external housing 4010. In one form a pneumatic block 4020 is supported by, or formed as part of the chassis 4016. An anti spillback valve 4160 may be provided between the pneumatic block 4020 and the humidifier 5000.

The RPT device 4000 may have an electrical power supply 4210, one or more input devices 4220, a central controller 4230, a therapy device controller 4240, a pressure generator 4140, one or more protection circuits 4250, memory 4260, transducers 4270, data communication interface 4280 and one or more output devices 4290. Electrical components may be mounted on a single Printed Circuit Board Assembly (PCBA) 4202. In an alternative form, the RPT device 4000 may include more than one PCBA 4202.

### 4.4.1 RPT Device Algorithms

As mentioned above, in some forms of the present technology, the central controller 4230 may be configured to implement one or more algorithms expressed as computer programs stored in a non-transitory computer readable storage medium, such as memory. The algorithms are generally grouped into groups referred to as modules.

In other forms of the present technology, some portion or all of the algorithms may be implemented by a controller of an external device such as the local external device or the remote external device. In such forms, data representing the input signals and/or intermediate algorithm outputs necessary for the portion of the algorithms to be executed at the external device may be communicated to the external device via the local external communication network or the remote external communication network. In such forms, the portion of the algorithms to be executed at the external device may be expressed as computer programs stored in a non-transitory computer readable storage medium accessible to the controller of the external device. Such programs configure the controller of the external device to execute the portion of the algorithms.

In such forms, the therapy parameters generated by the external device via the therapy engine module (if such forms part of the portion of the algorithms executed by the external device) may be communicated to the central controller to be passed to the therapy control module.

### 4.5 Air Circuit

An air circuit 4170 in accordance with an aspect of the present technology is a conduit or a tube constructed and arranged to allow, in use, a flow of air to travel between two components such as RPT device 4000 and the patient interface 3000 or 3800.

In particular, the air circuit 4170 may be in fluid connection with the outlet of the pneumatic block 4020 and the patient interface. The air circuit may be referred to as an air delivery tube. In some cases there may be separate limbs of the circuit for inhalation and exhalation. In other cases a single limb is used.

In some forms, the air circuit 4170 may comprise one or more heating elements configured to heat air in the air circuit, for example to maintain or raise the temperature of the air. The heating element may be in a form of a heated wire circuit, and may comprise one or more transducers, such as temperature sensors. In one form, the heated wire circuit may be helically wound around the axis of the air circuit 4170. The heating element may be in communication with a controller such as a central controller 4230. One example of an air circuit 4170 comprising a heated wire circuit is described in U.S. Pat. No. 8,733,349, which is incorporated herewithin in its entirety by reference.

### 4.5.1 Supplementary Gas Delivery

In one form of the present technology, supplementary gas, e.g. oxygen, 4180 is delivered to one or more points in the pneumatic path, such as upstream of the pneumatic block 4020, to the air circuit 4170, and/or to the patient interface 3000 or 3800.

### 4.6 Humidifier

### 4.6.1 Humidifier Overview

In one form of the present technology there is provided a humidifier 5000 (e.g. as shown in FIG. 5A) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

The humidifier 5000 may comprise a humidifier reservoir 5110, a humidifier inlet 5002 to receive a flow of air, and a humidifier outlet 5004 to deliver a humidified flow of air. In some forms, as shown in FIG. 5A and FIG. 5B, an inlet and an outlet of the humidifier reservoir 5110 may be the humidifier inlet 5002 and the humidifier outlet 5004 respectively. The humidifier 5000 may further comprise a humidifier base 5006, which may be adapted to receive the humidifier reservoir 5110 and comprise a heating element 5240. The reservoir 5110 comprises a conductive portion 5120 configured to allow efficient transfer of heat from the heating element 5240 to the volume of liquid in the reservoir 5110. The reservoir 5110 may comprise a water level indicator 5150.

In some arrangements, the humidifier reservoir dock 5130 may comprise a locking feature such as a locking lever 5135 configured to retain the reservoir 5110 in the humidifier reservoir dock 5130

### 4.7 Breathing Waveforms

FIG. 6A shows a model typical breath waveform of a person while sleeping. The horizontal axis is time, and the vertical axis is respiratory flow rate. While the parameter values may vary, a typical breath may have the following approximate values: tidal volume Vt 0.5L, inhalation time Ti 1.6s, peak inspiratory flow rate Qpeak 0.4 L/s, exhalation time Te 2.4s, peak expiratory flow rate Qpeak −0.5 L/s. The total duration of the breath, Ttot, is about 4s. The person typically breathes at a rate of about 15 breaths per minute (BPM), with Ventilation Vent about 7.5 L/min. A typical duty cycle, the ratio of Ti to Ttot, is about 40%.

### 4.8 Respiratory Therapy Modes

Various respiratory therapy modes may be implemented by the disclosed respiratory therapy system including CPAP therapy, Bi-level therapy and/or High Flow therapy.

### 4.9 Glossary

For the purposes of the present technology disclosure, in certain forms of the present technology, one or more of the following definitions may apply. In other forms of the present technology, alternative definitions may apply.

### 4.9.1 General

Air: In certain forms of the present technology, air may be taken to mean atmospheric air, and in other forms of the present technology air may be taken to mean some other combination of breathable gases, e.g. atmospheric air enriched with oxygen.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m²=1 millibar 0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.

Respiratory Pressure Therapy (RPT): The application of a supply of air to an entrance to the airways at a treatment pressure that is typically positive with respect to atmosphere.

Ventilator: A mechanical device that provides pressure support to a patient to perform some or all of the work of breathing.

### 4.9.1.1 Materials

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240.

Polycarbonate: a thermoplastic polymer of Bisphenol-A Carbonate.

### 4.9.1.2 Mechanical Properties

Resilience: Ability of a material to absorb energy when deformed elastically and to release the energy upon unloading.

Resilient: Will release substantially all of the energy when unloaded. Includes e.g. certain silicones, and thermoplastic elastomers.

Hardness: The ability of a material per se to resist deformation (e.g. described by a Young's Modulus, or an indentation hardness scale measured on a standardised sample size).
‘Soft’ materials may include silicone or thermo-plastic elastomer (TPE), and may, e.g. readily deform under finger pressure.
‘Hard’ materials may include polycarbonate, polypropylene, steel or aluminium, and may not e.g. readily deform under finger pressure.

Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

Floppy structure or component: A structure or component that will change shape, e.g. bend, when caused to support its own weight, within a relatively short period of time such as 1 second.

Rigid structure or component: A structure or component that will not substantially change shape when subject to the loads typically encountered in use. An example of such a use may be setting up and maintaining a patient interface in sealing relationship with an entrance to a patient's airways, e.g. at a load of approximately 20 to 30 cmH₂O pressure.

As an example, an I-beam may comprise a different bending stiffness (resistance to a bending load) in a first direction in comparison to a second, orthogonal direction. In another example, a structure or component may be floppy in a first direction and rigid in a second direction.

### 4.9.2 Respiratory Cycle

Apnea: According to some definitions, an apnea is said to have occurred when flow falls below a predetermined threshold for a duration, e.g. 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort, despite the airway being patent. A mixed apnea occurs when a reduction or absence of breathing effort coincides with an obstructed airway.

### 4.9.3 Anatomy

### 4.9.3.1 Anatomy of the Face

Ala: the external outer wall or “wing” of each nostril (plural: alar)

Alare: The most lateral point on the nasal ala.

Alar curvature (or alar crest) point: The most posterior point in the curved base line of each ala, found in the crease formed by the union of the ala with the cheek.

Auricle: The whole external visible part of the ear.

(nose) Bony framework: The bony framework of the nose comprises the nasal bones, the frontal process of the maxillae and the nasal part of the frontal bone.

(nose) Cartilaginous framework: The cartilaginous framework of the nose comprises the septal, lateral, major and minor cartilages.

Columella: the strip of skin that separates the nares and which runs from the pronasale to the upper lip.

Columella angle: The angle between the line drawn through the midpoint of the nostril aperture and a line drawn perpendicular to the Frankfort horizontal while intersecting subnasale.

Frankfort horizontal plane: A line extending from the most inferior point of the orbital margin to the left tragion. The tragion is the deepest point in the notch superior to the tragus of the auricle.

Glabella: Located on the soft tissue, the most prominent point in the midsagittal plane of the forehead.

Lateral nasal cartilage: A generally triangular plate of cartilage. Its superior margin is attached to the nasal bone and frontal process of the maxilla, and its inferior margin is connected to the greater alar cartilage.

Lip, lower (labrale inferius):

Lip, upper (labrale superius):

Greater alar cartilage: A plate of cartilage lying below the lateral nasal cartilage. It is curved around the anterior part of the naris. Its posterior end is connected to the frontal process of the maxilla by a tough fibrous membrane containing three or four minor cartilages of the ala.

Nares (Nostrils): Approximately ellipsoidal apertures forming the entrance to the nasal cavity. The singular form of nares is naris (nostril). The nares are separated by the nasal septum.

Naso-labial sulcus or Naso-labial fold: The skin fold or groove that runs from each side of the nose to the corners of the mouth, separating the cheeks from the upper lip.

Naso-labial angle: The angle between the columella and the upper lip, while intersecting subnasale.

Otobasion inferior: The lowest point of attachment of the auricle to the skin of the face.

Otobasion superior: The highest point of attachment of the auricle to the skin of the face.

Pronasale: the most protruded point or tip of the nose, which can be identified in lateral view of the rest of the portion of the head.

Philtrum: the midline groove that runs from lower border of the nasal septum to the top of the lip in the upper lip region.

Pogonion: Located on the soft tissue, the most anterior midpoint of the chin.

Ridge (nasal): The nasal ridge is the midline prominence of the nose, extending from the Sellion to the Pronasale.

Sagittal plane: A vertical plane that passes from anterior (front) to posterior (rear). The midsagittal plane is a sagittal plane that divides the body into right and left halves.

Sellion: Located on the soft tissue, the most concave point overlying the area of the frontonasal suture.

Septal cartilage (nasal): The nasal septal cartilage forms part of the septum and divides the front part of the nasal cavity.

Subalare: The point at the lower margin of the alar base, where the alar base joins with the skin of the superior (upper) lip.

Subnasal point: Located on the soft tissue, the point at which the columella merges with the upper lip in the midsagittal plane.

Supramenton: The point of greatest concavity in the midline of the lower lip between labrale inferius and soft tissue pogonion

### 4.9.3.2 Anatomy of the Skull

Frontal bone: The frontal bone includes a large vertical portion, the squama frontalis, corresponding to the region known as the forehead.

Mandible: The mandible forms the lower jaw. The mental protuberance is the bony protuberance of the jaw that forms the chin.

Maxilla: The maxilla forms the upper jaw and is located above the mandible and below the orbits. The frontal process of the maxilla projects upwards by the side of the nose, and forms part of its lateral boundary.

Nasal bones: The nasal bones are two small oblong bones, varying in size and form in different individuals; they are placed side by side at the middle and upper part of the face, and form, by their junction, the “bridge” of the nose.

Nasion: The intersection of the frontal bone and the two nasal bones, a depressed area directly between the eyes and superior to the bridge of the nose.

Occipital bone: The occipital bone is situated at the back and lower part of the cranium. It includes an oval aperture, the foramen magnum, through which the cranial cavity communicates with the vertebral canal. The curved plate behind the foramen magnum is the squama occipitalis.

Orbit: The bony cavity in the skull to contain the eyeball.

Parietal bones: The parietal bones are the bones that, when joined together, form the roof and sides of the cranium.

Temporal bones: The temporal bones are situated on the bases and sides of the skull, and support that part of the face known as the temple.

Zygomatic bones: The face includes two zygomatic bones, located in the upper and lateral parts of the face and forming the prominence of the cheek.

### 4.9.3.3 Anatomy of the Respiratory System

Diaphragm: A sheet of muscle that extends across the bottom of the rib cage. The diaphragm separates the thoracic cavity, containing the heart, lungs and ribs, from the abdominal cavity. As the diaphragm contracts the volume of the thoracic cavity increases and air is drawn into the lungs.

Larynx: The larynx, or voice box houses the vocal folds and connects the inferior part of the pharynx (hypopharynx) with the trachea.

Lungs: The organs of respiration in humans. The conducting zone of the lungs contains the trachea, the bronchi, the bronchioles, and the terminal bronchioles. The respiratory zone contains the respiratory bronchioles, the alveolar ducts, and the alveoli.

Nasal cavity: The nasal cavity (or nasal fossa) is a large air filled space above and behind the nose in the middle of the face. The nasal cavity is divided in two by a vertical fin called the nasal septum. On the sides of the nasal cavity are three horizontal outgrowths called nasal conchae (singular “concha”) or turbinates. To the front of the nasal cavity is the nose, while the back blends, via the choanae, into the nasopharynx.

Pharynx: The part of the throat situated immediately inferior to (below) the nasal cavity, and superior to the oesophagus and larynx. The pharynx is conventionally divided into three sections: the nasopharynx (epipharynx) (the nasal part of the pharynx), the oropharynx (mesopharynx) (the oral part of the pharynx), and the laryngopharynx (hypopharynx).

### 4.9.4 Patient Interface

Anti-asphyxia valve (AAV): The component or sub-assembly of a mask system that, by opening to atmosphere in a failsafe manner, reduces the risk of excessive CO₂ rebreathing by a patient.

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Frame: Frame will be taken to mean a mask structure that bears the load of tension between two or more points of connection with a headgear. A mask frame may be a non-airtight load bearing structure in the mask. However, some forms of mask frame may also be air-tight.

Headgear: Headgear will be taken to mean a form of positioning and stabilizing structure designed for use on a head. For example the headgear may comprise a collection of one or more struts, ties and stiffeners configured to locate and retain a patient interface in position on a patient's face for delivery of respiratory therapy. Some ties are formed of a soft, flexible, elastic material such as a laminated composite of foam and fabric.

Membrane: Membrane will be taken to mean a typically thin element that has, preferably, substantially no resistance to bending, but has resistance to being stretched.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.

Stiffener: A stiffener will be taken to mean a structural component designed to increase the bending resistance of another component in at least one direction.

Strut: A strut will be taken to be a structural component designed to increase the compression resistance of another component in at least one direction.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

Tie (noun): A structure designed to resist tension.

Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 4.9.5 Shape of Structures

Products in accordance with the present technology may comprise one or more three-dimensional mechanical structures, for example a mask cushion or an impeller. The three-dimensional structures may be bounded by two-dimensional surfaces. These surfaces may be distinguished using a label to describe an associated surface orientation, location, function, or some other characteristic. For example a structure may comprise one or more of an anterior surface, a posterior surface, an interior surface and an exterior surface. In another example, a seal-forming structure may comprise a face-contacting (e.g. outer) surface, and a separate non-face-contacting (e.g. underside or inner) surface. In another example, a structure may comprise a first surface and a second surface.

To facilitate describing the shape of the three-dimensional structures and the surfaces, we first consider a cross-section through a surface of the structure at a point, p. See FIG. 3B to FIG. 3F, which illustrate examples of cross-sections at point p on a surface, and the resulting plane curves. FIGS. 3B to 3F also illustrate an outward normal vector at p. The outward normal vector at p points away from the surface. In some examples we describe the surface from the point of view of an imaginary small person standing upright on the surface.

### 4.9.5.1 Curvature in One Dimension

The curvature of a plane curve at p may be described as having a sign (e.g. positive, negative) and a magnitude (e.g. 1/radius of a circle that just touches the curve at p).

Positive curvature: If the curve at p turns towards the outward normal, the curvature at that point will be taken to be positive (if the imaginary small person leaves the point p they must walk uphill). See FIG. 3B (relatively large positive curvature compared to FIG. 3C) and FIG. 3C (relatively small positive curvature compared to FIG. 3B). Such curves are often referred to as concave.

Zero curvature: If the curve at p is a straight line, the curvature will be taken to be zero (if the imaginary small person leaves the point p, they can walk on a level, neither up nor down). See FIG. 3D.

Negative curvature: If the curve at p turns away from the outward normal, the curvature in that direction at that point will be taken to be negative (if the imaginary small person leaves the point p they must walk downhill). See FIG. 3E (relatively small negative curvature compared to FIG. 3F) and FIG. 3F (relatively large negative curvature compared to FIG. 3E). Such curves are often referred to as convex.

### 4.9.5.2 Curvature of Two Dimensional Surfaces

A description of the shape at a given point on a two-dimensional surface in accordance with the present technology may include multiple normal cross-sections. The multiple cross-sections may cut the surface in a plane that includes the outward normal (a “normal plane”), and each cross-section may be taken in a different direction. Each cross-section results in a plane curve with a corresponding curvature. The different curvatures at that point may have the same sign, or a different sign. Each of the curvatures at that point has a magnitude, e.g. relatively small. The plane curves in FIGS. 3B to 3F could be examples of such multiple cross-sections at a particular point.

Principal curvatures and directions: The directions of the normal planes where the curvature of the curve takes its maximum and minimum values are called the principal directions. In the examples of FIG. 3B to FIG. 3F, the maximum curvature occurs in FIG. 3B, and the minimum occurs in FIG. 3F, hence FIG. 3B and FIG. 3F are cross sections in the principal directions. The principal curvatures at p are the curvatures in the principal directions.

Region of a surface: A connected set of points on a surface. The set of points in a region may have similar characteristics, e.g. curvatures or signs.

Saddle region: A region where at each point, the principal curvatures have opposite signs, that is, one is positive, and the other is negative (depending on the direction to which the imaginary person turns, they may walk uphill or downhill).

Dome region: A region where at each point the principal curvatures have the same sign, e.g. both positive (a “concave dome”) or both negative (a “convex dome”).

Cylindrical region: A region where one principal curvature is zero (or, for example, zero within manufacturing tolerances) and the other principal curvature is non-zero.

Planar region: A region of a surface where both of the principal curvatures are zero (or, for example, zero within manufacturing tolerances).

Edge of a surface: A boundary or limit of a surface or region.

Path: In certain forms of the present technology, ‘path’ will be taken to mean a path in the mathematical—topological sense, e.g. a continuous space curve from f(0) to f(1) on a surface. In certain forms of the present technology, a ‘path’ may be described as a route or course, including e.g. a set of points on a surface. (The path for the imaginary person is where they walk on the surface, and is analogous to a garden path).

Path length: In certain forms of the present technology, ‘path length’ will be taken to mean the distance along the surface from f(0) to f(1), that is, the distance along the path on the surface. There may be more than one path between two points on a surface and such paths may have different path lengths. (The path length for the imaginary person would be the distance they have to walk on the surface along the path).

Straight-line distance: The straight-line distance is the distance between two points on a surface, but without regard to the surface. On planar regions, there would be a path on the surface having the same path length as the straight-line distance between two points on the surface. On non-planar surfaces, there may be no paths having the same path length as the straight-line distance between two points. (For the imaginary person, the straight-line distance would correspond to the distance ‘as the crow flies’.)

### 4.9.5.3 Space Curves

Space curves: Unlike a plane curve, a space curve does not necessarily lie in any particular plane. A space curve may be closed, that is, having no endpoints. A space curve may be considered to be a one-dimensional piece of three-dimensional space. An imaginary person walking on a strand of the DNA helix walks along a space curve. A typical human left ear comprises a helix, which is a left-hand helix, see FIG. 3Q. A typical human right ear comprises a helix, which is a right-hand helix, see FIG. 3R. FIG. 3S shows a right-hand helix. The edge of a structure, e.g. the edge of a membrane or impeller, may follow a space curve. In general, a space curve may be described by a curvature and a torsion at each point on the space curve. Torsion is a measure of how the curve turns out of a plane. Torsion has a sign and a magnitude. The torsion at a point on a space curve may be characterised with reference to the tangent, normal and binormal vectors at that point.

Tangent unit vector (or unit tangent vector): For each point on a curve, a vector at the point specifies a direction from that point, as well as a magnitude. A tangent unit vector is a unit vector pointing in the same direction as the curve at that point. If an imaginary person were flying along the curve and fell off her vehicle at a particular point, the direction of the tangent vector is the direction she would be travelling.

Unit normal vector: As the imaginary person moves along the curve, this tangent vector itself changes. The unit vector pointing in the same direction that the tangent vector is changing is called the unit principal normal vector. It is perpendicular to the tangent vector.

Binormal unit vector: The binormal unit vector is perpendicular to both the tangent vector and the principal normal vector. Its direction may be determined by a right-hand rule (see e.g. FIG. 3P), or alternatively by a left-hand rule (FIG. 3O).

Osculating plane: The plane containing the unit tangent vector and the unit principal normal vector. See FIGS. 3O and 3P.

Torsion of a space curve: The torsion at a point of a space curve is the magnitude of the rate of change of the binormal unit vector at that point. It measures how much the curve deviates from the osculating plane. A space curve which lies in a plane has zero torsion. A space curve which deviates a relatively small amount from the osculating plane will have a relatively small magnitude of torsion (e.g. a gently sloping helical path). A space curve which deviates a relatively large amount from the osculating plane will have a relatively large magnitude of torsion (e.g. a steeply sloping helical path). With reference to FIG. 3S, since T2>T1, the magnitude of the torsion near the top coils of the helix of FIG. 3S is greater than the magnitude of the torsion of the bottom coils of the helix of FIG. 3S

With reference to the right-hand rule of FIG. 3P, a space curve turning towards the direction of the right-hand binormal may be considered as having a right-hand positive torsion (e.g. a right-hand helix as shown in FIG. 3S). A space curve turning away from the direction of the right-hand binormal may be considered as having a right-hand negative torsion (e.g. a left-hand helix).

Equivalently, and with reference to a left-hand rule (see FIG. 3O), a space curve turning towards the direction of the left-hand binormal may be considered as having a left-hand positive torsion (e.g. a left-hand helix). Hence left-hand positive is equivalent to right-hand negative. See FIG. 3T.

### 4.9.5.4 Holes

A surface may have a one-dimensional hole, e.g. a hole bounded by a plane curve or by a space curve. Thin structures (e.g. a membrane) with a hole, may be described as having a one-dimensional hole. See for example the one dimensional hole in the surface of structure shown in FIG. 3I, bounded by a plane curve.

A structure may have a two-dimensional hole, e.g. a hole bounded by a surface. For example, an inflatable tyre has a two dimensional hole bounded by the interior surface of the tyre. In another example, a bladder with a cavity for air or gel could have a two-dimensional hole. See for example the cushion of FIG. 3L and the example cross-sections therethrough in FIG. 3M and FIG. 3N, with the interior surface bounding a two dimensional hole indicated. In a yet another example, a conduit may comprise a one-dimension hole (e.g. at its entrance or at its exit), and a two-dimension hole bounded by the inside surface of the conduit. See also the two dimensional hole through the structure shown in FIG. 3K, bounded by a surface as shown.

### 4.10 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

1. A cushion module for a patient interface, the cushion module comprising:
a plenum chamber pressurisable to a therapeutic pressure of at least 6 cmH₂O above ambient air pressure, the plenum chamber configured to receive a flow of air at the therapeutic pressure for breathing by a patient;
a seal-forming structure constructed and arranged to form a seal with a region of the patient's face surrounding an entrance to the patient's airways, the seal-forming structure having at least one hole therein such that the flow of air at the therapeutic pressure is delivered to an entrance to the patient's nares and the patient's mouth, the seal-forming structure constructed and arranged to maintain said therapeutic pressure in the plenum chamber throughout the patient's respiratory cycle in use,
a chassis portion and a membrane portion together at least partially forming the plenum chamber, the membrane portion being attached to the chassis portion and forming the seal-forming structure, the chassis portion and membrane portion being formed separately;
wherein the membrane portion is attached to the chassis portion in an inverted position in which the membrane portion is positioned on an anterior side of the chassis portion, the membrane portion then being inverted to an in-use position in which the membrane portion is positioned on a posterior side of the chassis portion.

2. The cushion module of claim 1, wherein the membrane portion is attached to the chassis portion at or proximate a periphery of the chassis portion.

3. The cushion module of claim 1, wherein the membrane portion is formed at least partially from a textile material.

4. The cushion module of claim 3, wherein the membrane portion comprises an air-impermeable layer and a textile layer, the textile layer forming a patient-facing side of the membrane-portion.

5. The cushion module of claim 1, wherein the chassis portion is formed at least partially from a foam material.

6. The cushion module of claim 5, wherein the chassis portion comprises an air-impermeable layer provided to the foam material on at least one of the anterior side and the posterior side of the chassis portion.

7. The cushion module of claim 5, wherein the chassis portion comprises a textile layer on at least one of the anterior side and the posterior side of the chassis portion.

8. The cushion module of claim 1, wherein the chassis portion comprises a stiffening layer on an anterior side of the chassis portion.

9. The cushion module of claim 1, wherein the chassis portion comprises a nasal recess formed in the chassis portion and located proximate a pronasale region of the patient's nose in use, the nasal recess being recessed with respect to adjacent lateral portions of the chassis portion.

10. The cushion module of claim 9, wherein the nasal recess comprises a saddle portion formed in the chassis portion.

11. The cushion module of claim 9, wherein the membrane portion has a shape at the nasal recess which at least partially follows the shape of the chassis portion.

12. The cushion module of claim 9, wherein the membrane portion is slack at the nasal recess.

13. The cushion module of claim 12, wherein the cushion module is constructed and arranged to allow a pronasale region of the user's nose to lie in the nasal recess in use.

14. The cushion module of claim 12, wherein the cushion module is constructed and arranged to allow the pronasale of a patient with a long nose to protrude anteriorly of the anterior side of the chassis portion.

15. The cushion module of claim 9, wherein the membrane portion is taut at the nasal recess.

16. The cushion module of claim 15, wherein the membrane portion spans across the nasal recess from one lateral side of the nasal recess to the other lateral side of the nasal recess leaving space between the membrane portion and the chassis portion at the nasal recess.

17. The cushion module of claim 15, wherein the membrane portion is constructed and arranged to be deformed into or towards the shape of the nasal recess by the patient's nose in use.

18. The cushion module of claim 1, wherein the membrane portion comprises a first hole through which air can flow to both the patient's nares, in use, and wherein the membrane portion comprises a second hole through which air can flow to the patient's mouth, in use.

19. (canceled)

20. The cushion module of claim 1, wherein the chassis portion is formed by thermoforming or injection moulding.

21. (canceled)

22. (canceled)

23. (canceled)

24. (canceled)

25. The cushion module of claim 1, wherein the membrane portion is welded to the chassis portion.

26. (canceled)

27. The cushion module of claim 1, wherein the cushion module comprises headgear connection portions connected to the chassis portion, the headgear connection portions constructed and arranged to connect to a positioning and stabilising structure of the patient interface, wherein the headgear connection portions comprise a pair of superior headgear connection portions constructed and arranged to connect to superior strap portions of the positioning and stabilising structure of the patient interface and wherein the headgear connection portions comprise a pair of inferior headgear connection portions constructed and arranged to connect to inferior strap portions of the positioning and stabilising structure of the patient interface.

28. (canceled)

29. (canceled)

30. A patient interface comprising:
the cushion module of claim 1;
a vent to allow a continuous flow of gases exhaled by the patient from an interior of the plenum chamber of the cushion module to ambient, said vent being sized and shaped to maintain the therapeutic pressure in the plenum chamber in use;
a positioning and stabilising structure a positioning and stabilising structure configured to provide a force to hold the seal-forming structure in a therapeutically effective position on the patient's head in use;
wherein the patient interface is configured to allow the patient to breathe from ambient through their mouth in the absence of a flow of pressurised air through the plenum chamber inlet port, or the patient interface is configured to leave the patient's mouth uncovered in use.

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)
